(19) United States
(12) Patent Application Publication
(10) Pub. No.: US 2008/0167376 A1
(43) Pub. Date: Jul. 10, 2008

(54) Methods and products which utilize N-acyl-L-aspartic acid

(75) Inventors: David Bar-Or, Englewood, CO (US); Leonard T. Rael, Aurora, CO (US)

Correspondence Address: SHERIDAN ROSS PC, 1560 BROADWAY, SUITE 1200, DENVER, CO, 80202, US

(73) Assignee: DMI BIOSCIENCES, INC., Aurora, CO (US)

(21) Appl. No.: 11/966,293

(22) Filed: Dec. 28, 2007

Related U.S. Application Data
(62) Division of application No. 10/949,715, filed on Sep. 24, 2004, now abandoned.
(60) Provisional application No. 60/506,323, filed on Sep. 25, 2003.

(51) Int. Cl. A61K 31/195; A61P 29/00

(52) U.S. Cl. 514/561

(57) ABSTRACT

The invention provides therapeutic methods and products for the treatment of inflammation, inflammatory diseases and conditions, and proliferative diseases and conditions. The invention also provides methods and products for inhibiting inflammation in excised cells, tissues and organs. The invention further provides oral care methods and products for the treatment of the tissues of an animal's mouth. Finally, the invention provides personal care methods and products for the treatment of the skin of an animal. All of these methods and products utilize N-acyl-L-aspartic acid or an ester or pharmaceutically-acceptable salt thereof.

### CROSS-REFERENCE TO RELATED APPLICATION

This application is a divisional of U.S. patent application Ser. No. 10/949,715, filed Sep. 24, 2004, which claims the benefit of priority under 35 U.S.C. § 119(e) from provisional application Ser. No. 60/506,323, filed Sep. 25, 2003, the disclosure of which is incorporated herein by reference.

### FIELD OF THE INVENTION

This invention relates to methods and products which utilize N-acyl-L-aspartic acid or an ester or pharmaceutically-acceptable salt thereof. In a preferred embodiment, the invention relates to therapeutic methods and products for the treatment of inflammation, inflammatory diseases and conditions, and proliferative diseases and conditions. In another embodiment, the invention relates to methods and products for treating excised cells, tissues and organs. In yet another embodiment, the invention relates to oral care methods and products for the treatment of an animal's mouth. In a fourth embodiment, the invention relates to personal care methods and products, especially for the treatment of the skin of an animal.

### BACKGROUND

Inflammation is a cascade of events through which the body responds to a variety of injuries, infections and stresses. The inflammatory response is critical for stress response, fending off infections and healing wounds, but inflammation can also be damaging. Indeed, inflammation is an important component of the pathogenic process of many diseases and disorders. In addition, the presence of inflammation in many diseases, such as cancer, is indicative of a less favorable prognosis. Finally, in the extreme, inflammation may result in a life-threatening systemic response if not properly treated. Clearly, there is a continuing need for treatments for inflammation and inflammatory diseases and conditions.

Proliferative diseases and conditions include cancer and angiogenic diseases and conditions (e.g., tumor growth, tumor metastasis and macular degeneration). There is also a continuing need for treatments for proliferative diseases and conditions.

### SUMMARY OF THE INVENTION

In one embodiment, the invention provides a method of treating inflammation. The method comprises administering to an animal in need thereof an effective amount of a compound of formula I:

R¹—C(O)—NH—CH₂(CH₂—COOR²)—COOR² (I)

wherein:
R¹ is H, a lower alkyl or a lower alkyl substituted with a halogen atom; and
R², each of which may be the same or different, is H or an alkyl, cycloalkyl, aryl, alkylaryl or arylalkyl, each of which may optionally be substituted with a polar substitutent;
   or a pharmaceutically-acceptable salt thereof.

In another embodiment, the invention provides a method of treating an inflammatory disease or condition. The method comprises administering an effective amount of a compound of formula I or a pharmaceutically-acceptable salt thereof to an animal in need thereof.

In a further embodiment, the invention provides a method of treating a proliferative disease or condition. The method comprises administering an effective amount of a compound of formula I or a pharmaceutically-acceptable salt thereof to an animal in need thereof.

In another embodiment, the invention provides a method of treating a skin disease or condition. The method comprises administering an effective amount of a compound of formula I or a pharmaceutically-acceptable salt thereof to an animal in need thereof.

In a further embodiment, the invention provides a pharmaceutical composition. The composition comprises a compound of formula I or a pharmaceutically-acceptable salt thereof and a pharmaceutically-acceptable carrier.

In another embodiment, the invention provides a method of treating a cell, a tissue or an organ that has been removed from an animal. The method comprises contacting the cell, tissue or organ with a solution or medium containing an effective amount of a compound of formula I or a pharmaceutically-acceptable salt thereof.

In a further embodiment, the invention provides a solution or medium for contacting a cell, a tissue or an organ that has been removed from an animal. The solution or medium comprises a compound of formula I or a pharmaceutically-acceptable salt thereof.

In another embodiment, the invention provides a kit for contacting a cell, a tissue or an organ that has been removed from an animal with a compound of formula I or a pharmaceutically-acceptable salt thereof. The kit comprises a container holding the a compound of formula I or pharmaceutically-acceptable salt thereof.

In a further embodiment, a method of treating a tissue of an animal's mouth. The method comprises contacting the tissue with an effective amount of a compound of formula I or a pharmaceutically-acceptable salt thereof.

In another embodiment, the invention provides a method of treating a disease or condition of a mouth of an animal. The method comprises administering an effective amount of a compound of formula I or a pharmaceutically-acceptable salt thereof to the animal.

In a further embodiment, the invention provides a method of whitening one or more teeth of an animal. The method comprises contacting a tissue of the animal's mouth with an effective amount of a compound of formula I or a pharmaceutically-acceptable salt thereof.

In additional embodiments, the invention provides an oral care product comprising a compound of formula I or a pharmaceutically-acceptable salt thereof and a kit comprising the oral care product. The oral care product may be an oral care device or an oral care composition.

In a further embodiment, the invention provides a method of treating a portion of an animal's skin. The method comprises contacting the portion of the skin with an effective amount of a compound of formula I or a pharmaceutically-acceptable salt thereof.

In additional embodiments, the invention provides a personal care product comprising a compound of formula I or a pharmaceutically-acceptable salt thereof and a kit comprising the personal care product. The personal care product may be a personal care device or a personal care composition.

### BRIEF DESCRIPTION OF THE DRAWINGS

FIG. 1. Percent inhibition of prostaglandin E₂ (PGE₂) production in ionomycin-stimulated STTG cells. PGE₂ production was assayed in ionomycin-stimulated STTG cells treated with N-acetyl-L-aspartate (NAA), aspirin (Asp) or dexamethasone (Dex) using a PGE₂ enzyme immunoassay described in Example 1. Cells were pre-incubated for 1 hour with NAA, Asp or Dex and then stimulated with 1 μM ionomycin at 37° C., 10% CO₂ for 24 hours. Percent inhibition of PGE₂ production was measured compared to ionomycin-stimulated STTG cells in the absence of NAA, Asp or Dex. Data represent mean ±SD of three separate experiments. Asterisks indicate a significant difference between untreated, ionomycin-stimulated STTG cells and treated, ionomycin-stimulated STTG cells: *p<0.01. Iono=ionomycin.

FIG. 2. Effect of glutamate receptor antagonists and NAA on PGE₂ release in STTG cells stimulated with ionomycin. PGE₂ production was assayed in ionomycin-stimulated STTG cells treated with NAA or potential glutamate receptor antagonists (AP-4 and GDE) using a PGE₂ enzyme immunoassay described in Example 1. Cells were pre-incubated for 1 hour with NAA or the potential glutamate receptor antagonists and then cells were stimulated with 1 μM ionomycin at 37° C., 10% CO₂ for 24 hours. Percent inhibition of PGE₂ production was measured compared to ionomycin-stimulated STTG cells in the absence of NAA or potential glutamate receptor antagonists. Data represent mean ±SD of three separate experiments. Asterisks indicate a significant difference between untreated, ionomycin-stimulated STTG cells and treated, ionomycin-stimulated STTG cells: *p<0.01. AP-4=L-2-amino-4-phosphonobutyric acid, and GDE=L-glutamic acid diethyl ester.

FIG. 3. Representative Western blot for total COX-2 protein in IL-1β-stimulated STTG cells treated with NAA and aspirin. Cells were incubated for 24 hours at 37° C., 10% CO₂. Cells were dosed as follows: untreated (lane 1), 200 μM aspirin (lane 2), 10 mM NAA (lane 3), 1 ng/ml IL-1β (lane 4), 2 ng/ml IL-1β (lane 5), 1 ng/ml IL-1β+200 μM aspirin (lane 6), 2 ng/ml IL-1β+200 μM aspirin (lane 7), 1 ng/ml IL-1β+10 mM NAA (lane 8), or 2 ng/ml IL-1β+10 mM NAA (lane 9). Cells were lysed and COX-2 protein was immunoprecipitated overnight (1:500 goat anti-human COX-2) from 400 μg total lysate protein. Immunoprecipitate was loaded on 4-20% tris-glycine gel and transferred overnight onto nitrocellulose membrane. Total COX-2 protein was quantitated using a goat anti-human COX-2 antibody (1:100) and 1:5,000 rabbit anti-goat IgG. The membrane was visualized by chemiluminescence.

FIG. 4. Effect of NAA on NFκB levels in STTG cells stimulated with IL-1. Cells were dosed with NAA and immediately stimulated with 1 ng/ml IL-1 for 24 hours at 37° C., 10% CO₂. Cells were lysed, and 10 μg of total protein was assayed for activated NFκB levels using an ELISA-based method. Data represent mean ±SD of three separate experiments. Percentages are percent of inhibition of production of activated NFκB. Asterisks indicate a significant difference between untreated, IL-1β-stimulated STTG cells and NAA-treated, IL-1β-stimulated STTG cells: *p<0.005.

### DETAILED DESCRIPTION OF THE PRESENTLY PREFERRED EMBODIMENTS OF THE INVENTION

A. The Compounds

The invention provides methods and products which utilize a compound of formula I:

R¹—C(O)—NH—CH₂(CH₂—COOR²)—COOR² (I)

wherein:
R¹ is H, a lower alkyl or a lower alkyl substituted with a halogen atom; and
R², each of which may be the same or different, is H or an alkyl, cycloalkyl, aryl, alkylaryl or arylalkyl, each of which may optionally be substituted with a polar substitutent;
   or a pharmaceutically-acceptable salt of a compound of formula I. Highly preferred is N-acetyl-L-aspartic acid (NAA) or a pharmaceutically-acceptable salt of NAA.

“Alkyl” is used herein to mean a straight-chain or branched-chain saturated hydrocarbon, preferably containing 1-30 carbon atoms, more preferably containing 1-20 carbon atoms (e.g., methyl, ethyl, propyl, isopropyl, etc.).

“Aryl” is used herein to mean an aromatic group having at least one aromatic ring (e.g., phenyl).

“Alkylaryl” is used herein to mean an alkyl having an aryl attached thereto (e.g., —CH₂C₆H, or —CH₃CH(C₆H₅)CH₃).

“Arylalkyl” is used herein to mean an aryl having an alkyl attached thereto (e.g., —C₆H₄—CH₃).

“Cycloalkyl” is used herein to mean a saturated cyclic hydrocarbon containing at least one ring (e.g., cyclohexyl).

“Halogen atom” is used herein to mean bromine, chlorine, fluorine and iodine atoms. Preferred is substitution of a lower alkyl with 1-2 chlorine atoms or 1-3 fluorine atoms.

“Lower alkyl” is used herein to mean an alkyl containing 1-3 carbon atoms.

As used herein, “polar substituent” means a substituent that is typically charged in aqueous solutions (e.g., —OH, —COOH and —NH₂).

Compounds of formula I are available commercially from many sources or can be synthesized by methods well known in the art. Commercial sources of many of the compounds covered by formula I include Sigma-Aldrich Co., St. Louis, Mo., Rhodia Pharma Solutions, Cranbury, N.J., Spectrum Chemicals & Laboratory Products Inc., Gardena, Calif., BIOTREND Chemikalien GmbH, Cologne, Germany, Degussa A G, Marl, Germany, CHEMOS GmbH, Regenstauf, Germany, and DSL Chemicals (Shanghai) Co., Ltd., Shanghai, China, and The Lab Depot, Inc., Alpharetta, Ga. Methods useful for preparing compounds of formula I include those described in, e.g., Bodansky and Bodansky, The Practice of Peptide Synthesis, pages 63-66 (2nd ed., Springer-Verlag, 1994), Moore et al., Archives of biochemistry and Biophysics, 413(1):1-8 (May 2003), Liwschitz et al., J. Chem. Soc. C, 223-225 (1971) and U.S. Pat. Nos. 5,399,570, 5,756,465 and 6,200,969.

Pharmaceutically-acceptable salts of the compounds of formula I include conventional non-toxic salts, such as salts derived from inorganic or organic bases (e.g., the hydroxide, carbonate or bicarbonate of a pharmaceutically-acceptable metal cation). The salts are prepared in a conventional manner, e.g., by neutralizing the free acid form of the compound with a base.

B. Therapeutic Methods and Pharmaceutical Compositions

The invention provides therapeutic methods and pharmaceutical compositions for treating certain diseases and conditions. These methods and compositions utilize a compound of formula I or a pharmaceutically-acceptable salt thereof. As used herein, “treat” means to reduce (wholly or partially) the symptoms or severity of a disease or condition, including curing the disease or condition, or to prevent (wholly or partially) the disease or condition.

In particular, a compound of formula I or a pharmaceutically-acceptable salt thereof can be used to inhibit inflammation. Accordingly, a compound of formula I or a pharmaceutically-acceptable salt thereof can be used to treat inflammation. In a preferred embodiment, a compound of formula I or a pharmaceutically-acceptable salt thereof is used to treat inflammation of a mouth tissue, a mucous membrane, a portion of the skin, a portion of the respiratory system, or a portion of the gastrointestinal tract. In a more preferred embodiment, a compound of formula I or a pharmaceutically-acceptable salt thereof is used to treat inflammation of a mouth tissue, a mucous membrane or a portion of the skin.

“Inhibit” is used herein to mean to reduce (wholly or partially) or to prevent (wholly or partially).

“A” or “an” entity refers to one or more of that entity. For example, “a portion” refers to one or more portions.

A compound of formula I or a pharmaceutically-acceptable salt thereof can also be used to treat an inflammatory disease or condition. An inflammatory disease or condition is a disease or condition causing, caused by, involving, or exacerbated by, inflammation. In a preferred embodiment, a compound of formula I or a pharmaceutically-acceptable salt thereof is used to treat an inflammatory disease or condition of the mouth, the skin, the respiratory system or the gastrointestinal tract. In a more preferred embodiment, a compound of formula I or a pharmaceutically-acceptable salt thereof is used to treat an inflammatory disease or condition of the mouth or the skin. Specific inflammatory diseases and conditions that can be treated with a compound of formula I or a pharmaceutically-acceptable salt thereof include acute respiratory distress syndrome, allergies, arthritis, asthma, autoimmune diseases (e.g, multiple sclerosis), bronchitis, cancer, colitis, Crohn's disease, cystic fibrosis, emphysema, endocarditis, gingivitis, periodontitis, gastritis, infections (bacterial, viral, yeast, fungal and parasitic), inflammatory bowel disease, inflammatory skin diseases and conditions (see below), ischemia reperfusion, multiple organ dysfunction syndrome, multiple organ failure, nephritis, neurodegenerative diseases (e.g., Alzheimer's disease, amyotropic lateral sclerosis, Huntington's chorea, Parkinson's disease, senile dementia), pancreatitis, psoriasis, respiratory viral infections, sepsis, shock, systemic inflammatory response syndrome, trauma, ulcerative colitis and other inflammatory diseases and conditions.

In addition, to inflammation and inflammatory diseases and conditions, a compound of formula I or a pharmaceutically-acceptable salt thereof can be used to treat proliferative diseases and conditions. A proliferative disease or condition is a disease or condition causing, caused by, involving, or exacerbated by, proliferation of cells. Specific proliferative diseases and conditions that can be treated with a compound of formula I or a pharmaceutically-acceptable salt thereof include cancer, blood vessel proliferative disorders, mesangial cell proliferation disorders and fibrotic disorders.

Specific cancers treatable with a compound of formula I or a pharmaceutically-acceptable salt thereof include carcinomas, sarcomas, brain cancers, head and neck cancers, breast cancers, cervical cancers, ovarian cancers, uterine cancers, prostate cancers, stomach cancers, colon cancers, rectal cancers, pancreatic cancers, bladder cancers, thyroid cancers, hepatic cancers, lung cancers, bone cancers, skin cancers, blood cancers, lymphomas and leukemias.

Blood vessel proliferative disorders include angiogenic diseases and conditions. An angiogenic disease or condition is a disease or condition causing, caused by, involving, exacerbated by, or dependent on angiogenesis. Angiogenesis is the process of new blood vessel formation in the body, and a compound of formula I or a pharmaceutically-acceptable salt thereof will inhibit angiogenesis. Specific angiogenic diseases and conditions treatable in accordance with the invention include neoplastic diseases (e.g., tumors (e.g., tumors of the bladder, brain, breast, cervix, colon, rectum, kidney, lung, ovary, pancreas, prostate, stomach and uterus) and tumor metastasis), benign tumors (e.g., hemangiomas, acoustic neuromas, neurofibromas, trachomas, and pyrogenic granulomas), hypertrophy (e.g., cardiac hypertrophy induced by thyroid hormone), connective tissue disorders (e.g., rheumatoid arthritis and atherosclerosis), psoriasis, ocular angiogenic diseases (e.g., diabetic retinopathy, retinopathy of prematurity, macular degeneration, corneal graft rejection, neovascular glaucoma, retrolental fibroplasia, and rubeosis), cardiovascular diseases, cerebral vascular diseases, endometriosis, polyposis, obesity, diabetes-associated diseases, hemophiliac joints, and immune disorders (e.g., chronic inflammation, autoimmune diseases (e.g., multiple sclerosis) and transplant rejection). A compound of formula I or a pharmaceutically-acceptable salt thereof can also be used to inhibit the vascularization required for embryo implantation, thereby providing a method of birth control.

Mesangial cell proliferative disorders refer to disorders brought about by abnormal proliferation of mesangial cells. Mesangial cell proliferative disorders include renal diseases, such as glomerulonephritis, diabetic nephropathy, malignant nephrosclerosis, thrombotic microangiopathy syndromes and glomerulopathies.

Fibrotic disorders refer to the abnormal formation of extracellular matrices. Examples of fibrotic disorders include hepatic cirrhosis, pulmonary fibrosis (including idiopathic pulmonary fibrosis) and atherosclerosis.

Other proliferative disorders include hyperproliferative skin disorders, such as psoriasis, skin cancer and epidermal hyperproliferation. Psoriasis is characterized by inflammation, hyperproliferation of the epidermis and decreased differentiation of cells.

In a preferred embodiment of the invention, a compound of formula I or a pharmaceutically-acceptable salt thereof will be used to treat skin diseases and conditions. Skin diseases and conditions treatable with a compound of formula I or a pharmaceutically-acceptable salt thereof include an acne, a dermatitis, eczema, keratosis, elastosis, psoriasis, infections (e.g., measles and chicken pox), a burn, sunburn, an allergic reaction (e.g., rashes and hives), any other inflammatory disease or condition of the skin, and skin cancers.

In another preferred embodiment of the invention, a compound of formula I or a pharmaceutically-acceptable salt thereof will be used to treat diseases and conditions of the mouth. Mouth diseases and conditions treatable with a compound of formula I or a pharmaceutically-acceptable salt thereof include leukoplakia, lichen plannus, infections, other inflammatory diseases and conditions, and mouth cancers. Many other disease and conditions of the mouth, such as gingivits and periodontitis, will be typically be treated by, or under the supervision of, a dentist, and the treatment of these disease and conditions is described below in the section on oral care products and methods.

In yet another preferred embodiment of the invention, a compound of formula I or a pharmaceutically-acceptable salt thereof will be used to treat diseases and conditions of, or involving, the mucous membranes. Such diseases and conditions include allergies, infections and inflammatory diseases and conditions.

A compound of formula I or a pharmaceutically-acceptable salt thereof can be used to treat a disease or condition described above. To do so, it is administered to an animal in need of treatment for such a disease or condition. Preferably, the animal is a mammal, such as a rabbit, goat, dog, cat, horse or human. Most preferably, the animal is a human.

Effective dosage forms, modes of administration and dosage amounts for the a compound of formula I or a pharmaceutically-acceptable salt thereof may be determined empirically, and making such determinations is within the skill of the art. It is understood by those skilled in the art that the dosage amount will vary with the disease or condition to be treated, the severity of the disease or condition, the route(s) of administration, the rate of excretion of the compound, the duration of the treatment, the identify of any other drugs being administered to the animal, the age, size and species of the animal, and like factors known in the medical and veterinary arts. In general, a suitable daily dose of a compound of formula I or a pharmaceutically-acceptable salt thereof will be that amount of the compound which is the lowest dose effective to produce a therapeutic effect. However, the daily dosage will be determined by an attending physician or veterinarian within the scope of sound medical judgment. If desired, the effective daily dose may be administered as two, three, four, five, six or more sub-doses, administered separately at appropriate intervals throughout the day. Administration of a compound of formula I or a pharmaceutically-acceptable salt thereof should be continued until an acceptable response is achieved.

A compound of formula I or a pharmaceutically-acceptable salt thereof may be administered to an animal patient for therapy by any suitable route of administration, including orally, nasally, rectally, vaginally, parenterally (e.g., intravenously, intraspinally, intraperitoneally, subcutaneously, or intramuscularly), intracisternally, transdermally, intracranially, intracerebrally, and topically (including buccally and sublingually). A compound of formula I or a pharmaceutically-acceptable salt thereof is preferably administered by any type of local administration. “Local administration” is used herein to mean administration of a compound of formula I or a pharmaceutically-acceptable salt thereof by a route of administration and/or in a particular formulation that will provide a high dose of a compound of formula I or a pharmaceutically-acceptable salt thereof at or near the site of a disease or condition. Examples of local administration include topical administration (e.g., application of a lotion, cream or ointment containing a compound of formula I or a pharmaceutically-acceptable salt thereof to the skin to treat a skin disease or condition or administration of a compound of formula I or a pharmaceutically-acceptable salt thereof by means of an inhaler for treatment of a disease or condition of the respiratory system), nasal administration (e.g., administration of a compound of formula I or a pharmaceutically-acceptable salt thereof in a nose spray for treatment of a disease or condition of the nose), ocular administration (e.g, administration of a compound of formula I or a pharmaceutically-acceptable salt thereof in eye drops or by intra-ocular injection to treat an ocular disease or condition), vaginal administration, rectal administration, intra-tumor administration, and local oral administration (e.g., administration of a compound of formula I or a pharmaceutically-acceptable salt thereof in a rinse or syrup for treatment of a disease or condition of the mouth or throat or administration of a compound of formula I (preferably wherein R² is either a long alkyl (i.e., containing six or more carbon atoms) or is H or is substituted with polar substituents to prevent systemic absorption of the compound) or a pharmaceutically-acceptable salt thereof for treatment of a disease or condition of the gastrointestinal tract). Most preferred modes of local administration are local oral administration and topical administration.

While it is possible for a compound of formula I or a pharmaceutically-acceptable salt thereof to be administered alone, it is preferable to administer it as a pharmaceutical formulation (composition). The pharmaceutical compositions of the invention comprise a compound of formula I or a pharmaceutically-acceptable salt thereof as the active ingredient in admixture with one or more pharmaceutically-acceptable carriers and, optionally, with one or more other compounds, drugs or other materials. Each carrier must be “acceptable” in the sense of being compatible with the other ingredients of the formulation and not injurious to the animal. Pharmaceutically-acceptable carriers are well known in the art. Regardless of the route of administration selected, the compounds of the present invention are formulated into pharmaceutically-acceptable dosage forms by conventional methods known to those of skill in the art. See, e.g., Remington's Pharmaceutical Sciences.

Formulations of the invention suitable for oral administration may be in the form of capsules, cachets, pills, tablets, powders, granules or as a solution or a suspension in an aqueous or non-aqueous liquid, or an oil-in-water or water-in-oil liquid emulsions, or as an elixir or syrup, or as pastilles (using an inert base, such as gelatin and glycerin, or sucrose and acacia), and the like, each containing a predetermined amount of a compound of formula I or a pharmaceutically-acceptable salt thereof as an active ingredient. A compound of formula I or a pharmaceutically-acceptable salt thereof may also be administered as bolus, electuary or paste.

In solid dosage forms of the invention for oral administration (capsules, tablets, pills, dragees, powders, granules and the like), a compound of formula I or a pharmaceutically-acceptable salt thereof is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monosterate; (8) absorbents, such as kaolin and bentonite clay; (9) lubricants, such as talc, calcium stearate, magnesium stearate, solid polyethylene glycols, sodium lauryl sulfate, and mixtures thereof, and (10) coloring agents. In the case of capsules, tablets and pills, the pharmaceutical compositions may also comprise buffering agents. Solid compositions of a similar type may be employed as fillers in soft and hard-filled gelatin capsules using such excipients as lactose or milk sugars, as well as high molecular weight polyethylene glycols and the like.

A tablet may be made by compression or molding optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of a powdered compound of formula I or a pharmaceutically-acceptable salt thereof moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter. These compositions may also optionally contain opacifying agents and may be of a composition that they release the active ingredient only, or preferentially, in a certain portion of the gastrointestinal tract, optionally, in a delayed manner. Examples of embedding compositions which can be used include polymeric substances and waxes. The active ingredient can also be in microencapsulated form.

Liquid dosage forms for oral administration of a compound of formula I or a pharmaceutically-acceptable salt thereof include pharmaceutically-acceptable emulsions, microemulsions, solutions, suspensions, syrups and elixirs. In addition to the compound of formula I or pharmaceutically-acceptable salt thereof, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to a compound of formula I or a pharmaceutically-acceptable salt thereof, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the invention for rectal or vaginal administration may be presented as a suppository, which may be prepared by mixing a compound of formula I or a pharmaceutically-acceptable salt thereof with one or more suitable nonirritating excipients or carriers comprising, for example, cocoa butter, polyethylene glycol, a suppository wax or salicylate, and which is solid at room temperature, but liquid at body temperature and, therefore, will melt in the rectum or vaginal cavity and release the active compound. Formulations of the present invention which are suitable for vaginal administration also include pessaries, tampons, creams, gels, pastes, foams or spray formulations containing such carriers as are known in the art to be appropriate.

Dosage forms for the topical or transdermal administration of a compound of formula I or a pharmaceutically-acceptable salt thereof include powders, sprays, ointments, pastes, creams, lotions, gels, solutions, patches, drops and inhalants. A compound of formula I or a pharmaceutically-acceptable salt thereof may be mixed under sterile conditions with a pharmaceutically-acceptable carrier, and with any buffers, or propellants which may be required.

The ointments, pastes, creams and gels may contain, in addition to a compound of formula I or a pharmaceutically-acceptable salt thereof, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of formula I or a pharmaceutically-acceptable salt thereof, excipients such as lactose, talc, silicic acid, aluminum hydroxide, calcium silicates and polyamide powder or mixtures of these substances. Sprays can additionally contain customary propellants such as chlorofluorohydrocarbons and volatile unsubstituted hydrocarbons, such as butane and propane.

Transdermal patches have the added advantage of providing controlled delivery of a compound of formula I or a pharmaceutically-acceptable salt thereof to the body. Such dosage forms can be made by dissolving, dispersing or otherwise incorporating a compound of formula I or a pharmaceutically-acceptable salt thereof in a proper medium, such as an elastomeric matrix material. Absorption enhancers can also be used to increase the flux of a compound of formula I or a pharmaceutically-acceptable salt thereof across the skin. The rate of such flux can be controlled by either providing a rate-controlling membrane or dispersing a compound of formula I or a pharmaceutically-acceptable salt thereof in a polymer matrix or gel.

Pharmaceutical formulations include those suitable for administration by inhalation or insufflation or for nasal or intraocular administration. For administration to the upper (nasal) or lower respiratory tract by inhalation, a compound of formula I or a pharmaceutically-acceptable salt thereof is conveniently delivered from an insufflator, nebulizer or a pressurized pack or other convenient means of delivering an aerosol spray. Pressurized packs may comprise a suitable propellant such as dichlorodifluoromethane, trichlorofluoromethane, dichlorotetrafluoroethane, carbon dioxide, or other suitable gas. In the case of a pressurized aerosol, the dosage unit may be determined by providing a valve to deliver a metered amount.

Alternatively, for administration by inhalation or insufflation, the composition may take the form of a dry powder, for example, a powder mix of a compound of formula I or a pharmaceutically-acceptable salt thereof and a suitable powder base, such as lactose or starch. The powder composition may be presented in unit dosage form in, for example, capsules or cartridges, or, e.g., gelatin or blister packs from which the powder may be administered with the aid of an inhalator, insufflator or a metered-dose inhaler.

For intranasal administration, a compound of formula I or a pharmaceutically-acceptable salt thereof may be administered by means of nose drops or a liquid spray, such as by means of a plastic bottle atomizer or metered-dose inhaler. Typical of atomizers are the Mistometer (Wintrop) and Medihaler (Riker).

Drops, such as eye drops or nose drops, may be formulated with an aqueous or nonaqueous base also comprising one or more dispersing agents, solubilizing agents or suspending agents. Liquid sprays are conveniently delivered from pressurized packs. Drops can be delivered by means of a simple eye dropper-capped bottle or by means of a plastic bottle adapted to deliver liquid contents dropwise by means of a specially shaped closure.

Pharmaceutical compositions of this invention suitable for parenteral administrations comprise a compound of formula I or a pharmaceutically-acceptable salt thereof in combination with one or more pharmaceutically-acceptable sterile isotonic aqueous or non-aqueous solutions, dispersions, suspensions or emulsions, or sterile powders which may be reconstituted into sterile injectable solutions or dispersions just prior to use, which may contain antioxidants, buffers, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as wetting agents, emulsifying agents and dispersing agents. It may also be desirable to include isotonic agents, such as sugars, sodium chloride, and the like in the compositions. In addition, prolonged absorption of the injectable pharmaceutical form may be brought about by the inclusion of agents which delay absorption such as aluminum monosterate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug is accomplished by dissolving or suspending the drug in an oil vehicle.

Injectable depot forms are made by forming microencapsule matrices of the drug in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue. The injectable materials can be sterilized for example, by filtration through a bacterial-retaining filter.

The formulations may be presented in unit-dose or multi-dose sealed containers, for example, ampules and vials, and may be stored in a lyophilized condition requiring only the addition of the sterile liquid carrier, for example water for injection, immediately prior to use. Extemporaneous injection solutions and suspensions may be prepared from sterile powders, granules and tablets of the type described above.

A compound of formula I or a pharmaceutically-acceptable salt thereof can be administered alone or can be administered in combination with one or more other drugs, compounds or other materials. For instance, a compound of formula I or a pharmaceutically-acceptable salt thereof can be administered in combination with one or more additional anti-inflammatory compounds, including steroids, non-steroid anti-inflammatory compounds (e.g., aspirin, ibuprofen, etc.), and those anti-inflammatory compounds described in U.S. patent application Ser. Nos. 09/678,202, 09/922,234, and 10/186,168, and PCT applications WO 01/25265, WO 02/11676 and WO 02/64620, the complete disclosures of which are incorporated herein by reference.

C. Excised Cells, Tissues and Organs

A tissue or organ that has been removed from an animal can be contacted with a solution (e.g., by placing the tissue or organ in the solution and/or by perfusing an organ (e.g., a kidney) with the solution) containing an effective amount of a compound of formula I or a pharmaceutically-acceptable salt thereof to inhibit inflammation. Effective amounts of a compound of formula I or a pharmaceutically-acceptable salt thereof to include in such solutions can be determined empirically, and doing so is within the skill in the art. The harvested tissue or organ may subsequently be used for transplantation into a recipient or for research purposes (e.g., using a perfused liver to screen drugs). A compound of formula I or a pharmaceutically-acceptable salt thereof can be used alone or can be used in combination with other compounds, drugs or materials.

Many suitable solutions for use with tissues and organs are known into which a compound of formula I or a pharmaceutically-acceptable salt thereof can be incorporated. See, e.g., Hauet et al., J. Pharmacol. Exp. Ther., 297, 946-953 (2001); Hauet et al., J. Pharmacol. Exp. Ther., 292, 254-260 (2000); Dunphy et al., Am. J. Physiol., 276, H1591-H1598 (1999); Muhlbacher et al., Transplant Proc., 31, 2069-2070 (1999); Watts et al., J Mol. Cell. Cardiol., 31, 1653-1666 (1999); Suzer et al., Pharmacol. Res., 37, 97-101 (1998); Collins et al., Kidney Int'l, 42, Suppl. 38, S-197-S-202 (1992); Paller, Ren. Fail., 14, 257-260 (1992); Baron et al., J. Surg. Res., 51, 60-65 (1991); Hisatomi et al., Transplantation, 52, 754-755 (1991); Belzer et al., Transplantation, 45, 673-76 (1988); U.S. Pat. Nos. 4,798,824, 4,873,230, 4,879,283, 5,514,536, and 5,710,172; and PCT application WO 98/35551 (the disclosures of all of the foregoing are incorporated herein by reference).

For instance, a solution for flushing and cold storage of hearts is the Celsior™ solution (available from SangStat Medical Corp., Fremont, Calif.). Celsior™ solution contains:

TABLE A

| Component | Concentration | |
|---|---|---|
| Mannitol | 60 | mmol |
| Lactobionic Acid | 80 | mmol |
| Glutamic Acid | 20 | mmol |
| Histidine | 30 | mmol |
| Calcium Chloride | 0.25 | mmol |
| Potassium chloride | 15 | mmol |
| Magnesium Chloride | 13 | mmol |
| Sodium hydroxide | 100 | mmol |
| Reduced Glutathione | 3 | mmol |
| Water For Injection | Up to 1 | liter |

The accepted standard solution for preservation of kidneys is the University Of Wisconsin solution (available from Barr Laboratories under tradename ViaSpan®) which has the following composition:

TABLE B

| Component | Concentration | | Function |
|---|---|---|---|
| Raffinose | 30 | mM | Impermeant: suppression of hypothermic |
| | (17.83 | g/L) | cell swelling |
| Lactobionic acid | 100 | mM | Impermeant: suppression of hypothermic |
| | (35.83 | g/L) | cell swelling |
| Pentafraction (hydroxyethyl starch) | 50 | g/L | Colloid: reduction of interstitial edema and |
| | | | endothelial cell swelling |
| Glutathione | 3 | mM | Antioxidant |
| | (0.992 | g/L) | |
| Allopurinol | 1 | mM | Inhibition of xanthine oxidase activity and |
| | (0.136 | g/L) | purine metabolism/reduction of oxygen free |
| | | | radicals |
| Adenosine | 5 | mM | Restoration of high energy phosphate |
| | (1.34 | g/L) | |
| Potassium phosphate | 25 | mM | pH buffer: maintenance of intracellular |
| | (3.4 | g/L) | sodium and potassium concentrations: |
| | | | restoration of high energy phosphate |
| Magnesium sulfate | 5 | mM | Preservation of intracellular magnesium |
| | (1.23 | g/L) | concentration |
| Potassium hydroxide | 100 | mM | Maintenance of intracellular sodium and |
| | (5.61 | g/L) | potassium concentrations |
| Sodium hydroxide | 27 | mM | Maintenance of intracellular sodium and |
| | | | potassium concentrations |

Solution is pH adjusted to 7.4 with either sodium hydroxide or hydrochloric acid.
Final: Sodium = 29 mM; Potassium = 125 mM; mOsm/L = 320 ± 10
Immediately prior to use, to formulate the final solution, aseptically add: Penicillin G 200,000 units, regular insulin 40 units, and dexamethasone 16 mg.

A compound of formula I or a pharmaceutically-acceptable salt thereof could be used in either of these two solutions, variations of these solutions, or in one of the other numerous solutions known in the art or which will be developed. A compound of formula I or a pharmaceutically-acceptable salt thereof may be included in the solution or supplied separately (e.g., in lyophilized form) and added at the time of use.

Cells isolated from an animal can be stored or cultured in a medium containing an effective amount of a compound of formula I or a pharmaceutically-acceptable salt thereof. Many suitable media are known. Effective amounts of a compound of formula I or a pharmaceutically-acceptable salt thereof to include in the medium can be determined empirically, and doing so is within the skill in the art. A compound of formula I or a pharmaceutically-acceptable salt thereof may be included in the medium or supplied separately (e.g., in lyophilized form) and added at the time of use. The cells may be administered to a recipient in need thereof (e.g., for gene therapy) or may be used for research purposes.

The invention further provides a kit for contacting a cell, a tissue or organ that has been removed from an animal with a compound of formula I or a pharmaceutically-acceptable salt thereof. The kit is a packaged combination of one or more containers holding reagents and other items useful for preserving harvested cells, tissues or organs. The kit comprises a container holding a compound of formula I or a pharmaceutically-acceptable salt thereof. Suitable containers include bottles, bags, vials, test tubes, syringes, and other containers known in the art. For instance, the kit may comprise a vial containing a compound of formula I or a pharmaceutically-acceptable salt thereof. The kit may also contain other items which are known in the art and which may be desirable from a commercial and user standpoint, such as a container for the cells, tissue or organ, diluents, buffers, empty syringes, tubing, gauze pads, disinfectant solution, etc. The kits will also include instructions for using the kit to contact a cell, tissue or organ with a compound of formula I or a pharmaceutically-acceptable salt thereof contained in the kit.

D. Oral Care Products and Methods

A compound of formula I or a pharmaceutically-acceptable salt thereof can also be administered to an animal in oral care products. Oral care products include oral care compositions and oral care devices.

Oral care compositions of the invention include washes, rinses, gargles, solutions, drops, emulsions, suspensions, liquids, pastes, gels, ointments, creams, sprays, powders, tablets, gums, lozenges, mints, films, patches, and tooth whitening compositions. Oral care compositions of the invention include compositions intended for use by consumers and patients and compositions intended for use by dental professionals (e.g., dental hygienists, dentists and oral surgeons).

The oral care compositions of the invention will comprise a compound of formula I or a pharmaceutically-acceptable salt thereof as active ingredient in admixture with one or more pharmaceutically-acceptable carriers. The oral care compositions of the invention may also comprise one or more other acceptable ingredients, including other active compounds and/or other ingredients conventionally used in oral care compositions. Each carrier and ingredient must be “acceptable” in the sense of being compatible with the other ingredients of the formulation and not injurious to the animal.

Suitable ingredients, including pharmaceutically-acceptable carriers, for use in oral care compositions, and methods of making and using oral care compositions, are well known in the art. See, e.g., U.S. Pat. Nos. 4,847,283, 5,032,384, 5,043,183, 5,180,578, 5,198,220, 5,242,910, 5,286,479, 5,298,237, 5,328,682, 5,407,664, 5,466,437, 5,707,610, 5,709,873, 5,738,840, 5,817,295, 5,858,408, 5,876,701, 5,906,811, 5,932,193, 5,932,191, 5,951,966, 5,976,507, 6,045,780, 6,197,331, 6,228,347, 6,251,372, and 6,350,438, PCT applications WO 95/32707, WO 96/08232 and WO 02/13775, and EP applications 471,396, the complete disclosure of all of which are incorporated herein by reference. Conventional ingredients used in oral care compositions include water, alcohols, humectants, surfactants, thickening agents, abrasives, flavoring agents, sweetening agents, antimicrobial agents, anti-caries agents, anti-plaque agents, anti-calculus agents, pH-adjusting agents, and many others.

The water used in oral care compositions should preferably be of low ion content. It should also be free of organic impurities.

The alcohol must be nontoxic. Preferably the alcohol is ethanol. Ethanol is a solvent and also acts as an antibacterial agent and as an astringent.

Humectants suitable for use in oral care compositions include edible polyhydric alcohols, such as glycerol, sorbitol, xylitol, butylene glycol, polyethylene glycol, propylene glycol, mannitol and lactitol. Humectants help keep oral care compositions, such as pastes, from hardening upon exposure to air, give oral care compositions a moist feel to the mouth, and may impart desirable sweetness.

Surfactants include anionic, nonionic, amphoteric, zwitterionic and cationic synthetic detergents. Anionic surfactants include the water-soluble salts of alkyl sulfates having 8-20 carbon atoms in the alkyl radical (such as sodium alkyl sulfate), the water-soluble salts of sulfonated monoglycerides of fatty acids having from 8-20 carbon atoms (such as sodium lauryl sulfate and sodium coconut monoglyceride sulfonates), sarcosinates (such as sodium and potassium salts of lauroyl sarcosinate, myristoyl sarcosinate, palmitoyl sarcosinate, stearoyl sarcosinate and oleoyl sarcosinate), taurates, higher alkyl sulfoacetates (such as sodium lauryl sulfoacetate), isethionates (such as sodium lauroyl isethionate), sodium laureth carboxylate, sodium dodecyl benezesulfonate, and mixtures of the foregoing. Preferred are the sarcosinates since they inhibit acid formation in the mouth due to carbohydrate breakdown. Nonionic surfactants include poloxamers (sold under the tradename Pluronic), polyoxyethylene sorbitan esters (sold under the tradename Tween), fatty alcohol ethoxylates, polyethylene oxide condensates of alkyl phenols, products derived from the condensation of ethylene oxide with fatty acids, fatty alcohols, fatty amides, polyhydric alcohols, and polypropyleneoxide, ethylene oxide condensates of aliphatic alcohols, long-chain tertiary amine oxides, long-chain tertiary phosphine oxides, long-chain dialkyl sulfoxides, and mixtures of such materials. Amphoteric surfactants include betaines (such as cocamidopropylbetaine), derivatives of aliphatic secondary and tertiary amines in which the aliphatic radical can be a straight or branched chain and wherein one of the aliphatic substituents contains about 8-18 carbon atoms and one contains an anionic water-solubilizing group (such as carboxylate, sulfonate, sulfate, phosphate or phosphonate), and mixtures of such materials. Zwitterionic surfactants include derivatives of aliphatic quaternary ammonium, phosphonium and sulfonium compounds in which the aliphatic radical can be a straight or branched chain and wherein one of the aliphatic substituents contains about 8-18 carbon atoms and one contains an anionic water-solubilizing group (such as carboxy, sulfonate, sulfate, phosphate or phosphonate). Cationic surfactants include aliphatic quaternary ammonium compounds having one long alkyl chain containing about 8-18 carbon atoms (such as lauryl trimethylammonium chloride, cetylpyridinium chloride, cetyltrimethylammonium bromide, diisobuytylphenoxyethyldimethylbenzylammonium chloride, coconut alkyltrimethylammonium nitrite, cetylpyridinium fluoride). Certain cationic surfactants can also act as antimicrobials.

Thickening agents include carboxyvinyl polymers, polyvinylpyrrolidone, polyacrylates, carrageenan, cellulose derivatives (e.g., hydroxypropyl cellulose, hydroxypropyl methyl cellulose, methyl cellulose, and hydroxyethyl cellulose), laponite, water-soluble salts of cellulose ethers (such as sodium carboxymethylcellulose and sodium carboxymethyl hydroxyethyl cellulose), natural gums (such as gum karaya, xanthan gum, gum arabic and gum tragacanth), polymeric polyether compounds (such as polyethylene oxide and polypropylene oxide), homopolymers of acrylic acid crosslinked with an alkyl ether of pentaerythritol, alkyl ether of sucrose, carbomers (sold under the tradename Carbopolt), starch, copolymers of lactide and glycolide monomers (the copolymer having an average molecular weight of about 1,000-120,000), colloidal magnesium aluminum silicate and finely divided silica. Thickening agents will be added in amounts sufficient to give a desired consistency to an oral care composition.

Abrasives include silicas (including gels and precipitates), aluminas, calcium carbonates, calcium phosphates, dicalcium phosphates, tricalcium phosphates, hydroxyapatites, calcium pyrophosphates, trimetaphosphates, insoluble polymetaphosphates (such as insoluble sodium polymetaphosphate and calcium polymetaphosphate), magnesium carbonates, magnesium oxides, resinous abrasive materials (such as particulate condensation products of urea and formaldehyde), particulate thermosetting polymerized resins (suitable resins include melamines, phenolics, ureas, melamine-ureas, melamine-formaldehydes, urea-formaldehydes, melamine-urea-formaldehydes, cross-linked epoxides and cross-linked polyesters), and combinations of the foregoing. Silica abrasives are preferred because they provide excellent dental cleaning and polishing performance without unduly abrading tooth enamel or dentine.

Flavoring agents include peppermint, oil, spearmint oil, wintergreen oil, clove, menthol, dihydroanethole, estragole, methyl salicylate, eucalyptol, cassia, 1-menthyl acetate, sage, eugenol, parsley oil, menthone, oxanone, alpha-irisone, alpha-ionone, anise, marjoram, lemon, orange, propenyl guaethol, cinnamon, vanillin, ethyl vanillin, thymol, linalool, limonene, isoamylacetate, benzaldehyde, ethylbutyrate, phenyl ethyl alcohol, sweet birch, cinnamic aldehyde, cinnamaldehyde glycerol acetal (known as CGA), and mixtures of the foregoing.

Sweetening agents include sucrose, glucose, saccharin, dextrose, levulose, lactose, mannitol, sorbitol, fructose, maltose, xylitol, saccharin salts, thaumatin, aspartame, D-tryptophan, dihydrochalcones, acesulfame, cyclamate salts, and mixtures of the foregoing.

In addition to the flavoring and sweetening agents, the oral care compositions may include coolants, salivating agents, warming agents and numbing agents as optional ingredients. Coolants include carboxamides, menthol, paramenthan carboxamides, isopropylbutanamide, ketals, diols, 3-1-menthoxypropane-1,2-diol, menthone glycerol acetal, menthyl lactate, and mixtures thereof. Salivating agents include Jambut (manufactured by Takasago). Warming agents include capsicum and nicotinate esters (such as benzyl nicotinate). Numbing agents include benzocaine, lidocaine, clove bud oil and ethanol.

Antibacterial and anti-plaque agents include triclosan, sanguinarine and sanguinaria, quaternary ammonium compounds, cetylpyridinium chloride, tetradecylpyridinium chloride and N-tetradecyl-4-ethylpyridinium chloride, benzalkonium chloride, bisquanides, chlorhexidine, chlorhexidine digluconate, hexetidine, octenidine, alexidine, halogenated bisphenolic compounds, 2,2′-methylenebis-(4-chloro-6-bromophenol), 5-chloro-2-(2,4-dichlorophenoxy)-phenol, salicylanilide, domiphen bromide, delmopinol, octapinol, other piperadino derivatives, nicin, zinc stannous ion agents, antibiotics (such as augimentin, amoxicillin, tetracycline, doxycycline, minocycline, and metronidazole), analogs and salts of the foregoing, and mixtures of the foregoing.

Anti-caries agents include sodium fluoride, stannous fluoride, potassium fluoride, amine fluorides, indium fluoride, sodium monofluorophosphate, calcium lactate, calcium glycerophosphates, strontium salts, and strontium polyacrylates.

Anti-calculus agents include pyrophosphate salts such as dialkali metal pyrophosphate salts and tetraalkali metal pyrophosphate salts (e.g., disodium dihydrogen pyrophosphate, tetrasodium pyrophosphate and tetrapotassium pyrophosphate, in their hydrated and unhydrated forms). Other anti-calculus agents which can be used instead of, or in addition to, the pyrophosphate salts include synthetic anionic polymers (such as polyacrylates and copolymers of maleic anhydride or acid and methyl vinyl ether), polyaminopropane sulfonic acid, zinc citrate trihydrate, polyphosphates (such as tripolyphosphate and hexametaphosphate), polyphosphonates (such as disodium ethane-1-hydroxy-1,1-diphosphonate (EHDP), methanedisphosphonic acid, and 2-phosphonobutane-1,2,4-tricarboxylic acid), and polypeptides (such as polyaspartic acid and polyglutamic acid).

The pH of the oral care compositions of the invention should preferably not be acidic. Thus, the pH of the oral care compositions of the invention should be greater than about 6.5, preferably from about 7.0 to about 8.5, more preferably from about 7.2 to about 7.6. Thus, a pH-adjusting agent and/or a buffering agent or agents may need to be included in the oral care compositions. The pH-adjusting agent may be any compound or mixture of compounds that will achieve the desired pH. Suitable pH-adjusting agents include organic and inorganic acids and bases, such as benzoic acid, citric acid, potassium hydroxide, and sodium hydroxide. Buffering agents include acetate salts, borate salts, carbonate salts, bicarbonate salts (e.g., an alkali metal bicarbonate, such as sodium bicarbonate (also known as baking soda)), gluconates, tartrates, sulfates, citrates (such as sodium citrate), benzoate salts, nitrate salts (such as sodium and potassium nitrate), and combinations of the foregoing as needed to achieve and maintain the desired pH.

In addition to a compound of formula I or a pharmaceutically-acceptable salt thereof, the oral care compositions of the invention may include one or more additional anti-inflammatory agents, antioxidants and/or metal-binding compounds.

Suitable anti-inflammatory agents include ibuprofen, flurbiprofen, ketoprofen, aspirin, kertorolac, naproxen, indomethacin, piroxicam, meclofenamic acid, steroids, and mixtures of the foregoing.

Suitable antioxidants include superoxide dismutase, catalase, glutathione peroxidase, ebselen, glutathione, cysteine, N-acetyl cysteine, penicillamine, allopurinol, oxypurinol, ascorbic acid, α-tocopherol, Trolox (water-soluble α-tocopherol), vitamin A, β-carotene, fatty-acid binding protein, fenozan, probucol, cyanidanol-3, dimercaptopropanol, indapamide, emoxipine, dimethyl sulfoxide, and others. See, e.g., Das et al., Methods Enzymol., 233, 601-610 (1994); Stohs, J. Basic Clin. Physiol. Pharmacol., 6, 205-228 (1995).

Suitable metal-binding compounds include metal-binding peptide and/or non-peptide chelators. Metal-binding peptides and non-peptide chelators are known in the art. Preferred are those metal-binding peptides and non-peptide chelators described in PCT applications WO 01/25265 and WO 02/64620, the complete disclosures of which are incorporated herein by reference. Additional metal-binding compounds are polyethylenepolyamines, such as tetraethylenetriamine (trientine). See co-pending U.S. application Ser. No. 10/840,943 and PCT application number PCT/US04/14208, both filed May 7, 2004.

The oral care compositions of the invention may advantageously contain a protease inhibitor for an additional therapeutic effect (certain proteases are involved in inflammatory processes and others have been implicated in tissue breakdown in the mouth). Suitable protease inhibitors include metalloproteinase and serine protease inhibitors, such as those described in U.S. Pat. Nos. 6,403,633, 6,350,438, 6,066,673, 5,622,984, and 4,454,338, the complete disclosures of which are incorporated herein by reference.

Many other ingredients are known that may be incorporated into oral care compositions. These include suspending agents (such as a polysaccharide—see U.S. Pat. No. 5,466,437), polymeric compounds which can enhance the delivery of active ingredients (such as copolymers of polyvinylmethylether with maleic anhydride and those delivery enhancing polymers described in DE 942,643 and U.S. Pat. No. 5,466,437), materials which allow for a strong and continuing adherence of the oral care composition to the tissues of the mouth, thereby providing for a protracted topical therapeutic effect (such as natural gums, plant extracts, animal extracts (e.g., gelatin), natural and synthetic polymers, and starch derivatives; see, e.g., U.S. Pat. Nos. 5,032,384, 5,298,237, and 5,466,437), oils, waxes, silicones, coloring agents (such as FD&C dyes), color change systems, preservatives (such as methylparaben, propylparaben, and sodium benzoate), opacifying agents (such as titanium dioxide), plant extracts, solubilizing agents (such as propylene glycol; see, e.g., U.S. Pat. No. 5,466,437), enzymes (such as dextranase and/or mutanase, amyloglucosidase, glucose oxidase with lactoperoxidase, and neuraminidases), synthetic or natural polymers, tooth whitening agents (such from about 0.1% to about 10% by weight of a peroxygen compound; see additional discussion of tooth whitening compositions below), an alkali metal bicarbonate (such as sodium bicarbonate (also known as baking soda), generally present at from about 0.01% to about 30% by weight), desensitizers (such as potassium salts (e.g., potassium nitrate, potassium citrate, potassium chloride, potassium tartrate, potassium bicarbonate, and potassium oxalate) and strontium salts), analgesics (such as lidocaine or benzocaine), anti-fungal agents, antiviral agents, etc.

It will be appreciated that a wide variety of different oral care compositions can be prepared utilizing the above described ingredients and other ingredients known in the art or which will be developed. It is within the skill in the art to chose appropriate ingredients and combinations of ingredients and to determine an effective amount of a compound of formula I or a pharmaceutically-acceptable salt thereof to include in a particular oral care composition, given the knowledge in the art and the guidance provided herein.

What follows are a few examples of oral care compositions into which a compound of formula I or a pharmaceutically-acceptable salt thereof could be incorporated. It will be understood by those skilled in the art that additional types of oral care compositions and additional oral care compositions having different ingredients and/or different amounts of ingredients can be prepared utilizing the knowledge and skill in the art and the guidance provided herein.

Dentrifices include toothpastes, tooth gels, tooth powders and liquid dentrifices. Toothpastes and tooth gels generally include a dental abrasive, a surfactant, a thickening agent, a humectant, a flavoring agent, a sweetening agent, a coloring agent and water. Toothpastes and tooth gels may also include opacifying agents, anti-caries agents, anti-calculus agents, tooth whitening agents, and other optional ingredients. Typically, a toothpaste or tooth gel will contain from about 5% to about 70%, preferably from about 10% to about 50%, of an abrasive, from about 0.5% to about 10% of a surfactant, from about 0.1% to about 10% of a thickening agent, from about 10% to about 80% of a humectant, from about 0.04% to about 2% of a flavoring agent, from about 0.1% to about 3% of a sweetening agent, from about 0.01% to about 0.5% of a coloring agent, from about 0.05% to about 0.3% of an anti-caries agent, from about 0.1% to about 13% of an anti-calculus agent, and from about 2% to about 45% water. Tooth powders of course contain substantially all non-liquid components and typically contain from about 70% to about 99% abrasive. Liquid dentrifices may comprise water, ethanol, a humectant, a surfactant, a thickening agent, an abrasive (if an abrasive is included, a suspending agent (e.g., a high molecular weight polysaccharide) must be included; see U.S. Pat. No. 5,466,437), an antibacterial agent, an anti-caries agent, a flavoring agent and a sweetening agent. A typical liquid dentrifice will comprise from about 50% to about 85% water, from about 0.5% to about 20% ethanol, from about 10% to about 40% of a humectant, from about 0.5% to about 5% of a surfactant, from about 0.1% to about 10% of a thickening agent, and may contain from about 10% to about 20% of an abrasive, from about 0.3% to about 2% of a suspending agent, from about 0.05% to about 4% of an antibacterial agent, from about 0.0005% to about 3% of an anti-caries agent, from about 0.1% to about 5% of a flavoring agent, and from about 0.1% to about 5% of a sweetening agent.

Gels include dentrifice gels (see description above), non-abrasive gels and subgingival gels. Non-abrasive gels and subgingival gels generally include a thickening agent, a humectant, a flavoring agent, a sweetening agent, a coloring agent, and water. Such gels may also include one or more anti-caries agents and/or anti-calculus agents. Typically, such a gel will contain from about 0.1% to about 20% of a thickening agent, from about 10% to about 55% of a humectant, from about 0.04% to about 2% of a flavoring agent, from about 0.1% to about 3% of a sweetening agent, from about 0.01% to about 0.5% of a coloring agent, and the balance water. Such gels may also contain from about 0.05% to about 0.3% of an anti-caries agent and from about 0.1% to about 13% of an anti-calculus agent.

Creams generally include a thickening agent, a humectant and a surfactant, and may include a flavoring agent, a sweetening agent, a coloring agent. Typically, a cream will contain from about 0.1% to about 30% of a thickening agent, from about 0% to about 80% of a humectant, from about 0.1% to about 5% of a surfactant, from about 0.04% to about 2% of a flavoring agent, from about 0.1% to about 3% of a sweetening agent, from about 0.01% to about 0.5% of a coloring agent, and from about 2% to about 45% of water.

Ointments suitable for oral use are described in, e.g., U.S. Pat. Nos. 4,847,283, 5,855,872 and 5,858,408, the complete disclosures of which are incorporated herein by reference. Ointments generally include one or more of the following: fats, oils, waxes, parafins, silicones, plastibase, alcohols, water, humectants, surfactants, thickening agents, talc, bentonites, zinc oxide, aluminum compounds, preservatives, antiviral compounds, and other ingredients. For instance, the ointment may comprise from about 80% to about 90% petrolatum and from about 10% to about 20% ethanol or propylene glycol. As another example, the ointment may comprise about 10% petrolatum, about 9% lanolin, about 8% talc, about 32% cod liver oil, and about 40% zinc oxide. As a third example, the ointment may comprise from about 30% to about 45% water, from about 10% to about 30% oil (e.g., petrolatum or mineral oil), from about 0.1% to about 10% emulsifier (e.g., wax NF), from about 2% to about 20% humectant (e.g., propylene glycol), from about 0.05% to about 2% preservatives (e.g., methyl paraben and propyl paraben), and from about 10% to about 40% sterol alcohol.

Mouthwashes, rinses, gargles and sprays generally include water, ethanol, and/or a humectant, and preferably also include a surfactant, a flavoring agent, a sweetening agent, and a coloring agent, and may include a thickening agent and one or more anti-caries agents and/or anti-calculus agents. A typical composition contains from about 0% to about 80% of a humectant, from about 0.01% to about 7% of a surfactant, from about 0.03% to about 2% of a flavoring agent, from about 0.005% to about 3% of a sweetening agent, from about 0.001% to about 0.5% of a coloring agent, with the balance being water. Another typical composition contains from about 5% to about 60%, preferably from about 5% to about 20%, ethanol, from about 0% to about 30%, preferably from about 5% to about 20%, of a humectant, from about 0% to about 2% emulsifying agents, from about 0% to about 0.5% of a sweetening agent, from about 0% to about 0.3% of a flavoring agent, and the balance water. A further typical composition contains from about 45% to about 95% water, from about 0% to about 25% ethanol, from about 0% to about 50% of a humectant, from about 0.1% to about 7% of a surfactant, from about 0.1% to about 3% of a sweetening agent, from about 0.4% to about 2% of a flavoring agent, and from about 0.001% to about 0.5% of a coloring agent. These compositions may also comprise from about 0.05% to about 0.3% of an anti-caries agent, and from about 0.1% to about 3% of an anti-calculus agent

Solutions generally include water, a preservative, a flavoring agent, and a sweetening agent, and may include a thickening agent and/or a surfactant. Typically, solutions contain from about 85% to about 99% water, from about 0.01% to about 0.5% of a preservative, from about 0% to about 5% of a thickening agent, from about 0.04% to about 2% of a flavoring agent, from about 0.1% to about 3% of a sweetening agent, and from about 0% to about 5% of a surfactant.

Lozenges and mints generally include a base, a flavoring agent and a sweetening agent. The base may be a candy base (hard sugar candy), glycerinated gelatin or a combination of sugar with sufficient mucilage to give it form. See U.S. Pat. No. 6,350,438 and Remington, The Science And Practice Of Pharmacy, 19th edition (1995). Lozenge compositions also typically include one or more fillers (e.g., a compressible sugar) and lubricants.

Chewing gums, chewable tablets and chewable lozenges are described in U.S. Pat. Nos. 6,471,991, 6,296,868, 6,146,661, 6,060,078, 5,869,095, 5,709,873, 5,476,647, and 5,312,626, PCT applications WO 84/04453 and WO 99/02137, and Lieberman et al., Pharmaceutical Dosage Forms, 2^{nd} ed. (1990), the complete disclosures of which are incorporated here in by reference.

As one example, a compressed chewable tablet comprises a water-disintegratable, compressible carbohydrate (such as mannitol, sorbitol, maltitol, dextrose, sucrose, xylitol, lactose and mixtures thereof), a binder (such as cellulose, cellulosic derivatives, polyvinyl pyrrolidone, starch, modified starch and mixtures thereof), and, optionally, a lubricant (such as magnesium stearate, stearic acid, talc, and waxes), sweetening, coloring and flavoring agents, a surfactant, a preservative, and other ingredients. All of the ingredients, including a compound of formula I or a pharmaceutically-acceptable salt thereof, are dry blended and compressed into a tablet.

As another example, a chewable tablet may comprise a core surrounded by an outer layer wrapping the core. The core may comprise a compound of formula I or a pharmaceutically-acceptable salt thereof and, optionally, other active ingredients in a jelly base or a chewable base. The outer layer may be a chewable base. The jelly base may comprise pectin, sorbitol, maltitol, isomalt, liquid glucose, sugar, citric acid and/or a flavoring agent. The chewable base of the core or outer layer may be a gum, soft candy, nougat, caramel or hard candy. The tablets are formed by extrusion of the core and outer layer to form a rope, followed by cutting the rope into tablets.

Chewing gum compositions generally include a gum base, a flavoring agent and a sweetening agent. Suitable gum bases include jelutong, rubber, latex, chicle, and vinylite resins, desirably with conventional plasticizers or softeners. Plasticizers include triacetin, acetyl tributyl citrate, diethyl sebacetate, triethyl citrate, dibutyl sebacetate, dibutyl succinate, diethyl phthalate and acetylated monoglycerides. Typically, chewing gum compositions contain from about 50% to about 99% gum base, from about 0.4% to about 2% of a flavoring agent and from about 0.01% to about 20% of a sweetening agent. The compound of formula I or a pharmaceutically-acceptable salt thereof and other active ingredients may be incorporated into a gum base by, e.g., stirring them into a warm gum base or coating them onto the outer surface of the gum base.

Films and sheets, and gels which form solids in the mouth, made of lactide/glycolide copolymers are described in U.S. Pat. Nos. 5,198,220, 5,242,910 and 6,350,438. Another polymer film suitable for use in the mouth is described in PCT application WO 95/32707. Patches that adhere to hard dental surfaces, such as teeth and dentures, and which degrade in the mouth, are described in U.S. Pat. No. 6,197,331. All of these materials slowly release active agents contained in them into the mouth. Other compositions (including pastes, gels, ointments, liquids and films) providing for slow release of active agents are also known. See, e.g., U.S. Pat. Nos. 5,032,384, 5,298,237, 5,466,437, 5,709,873, and 6,270,781.

Tooth whitening compositions will comprise a tooth whitening agent. Tooth whitening agents include peroxides, percarbonates and perborates of the alkali and alkaline earth metals or complex compounds containing hydrogen peroxide. Tooth whitening agents also include peroxide salts of the alkali or alkaline earth metals. The most commonly used tooth whitening agent is carbamide peroxide. Other commonly used tooth whitening agents are hydrogen peroxide, peroxyacetic acid and sodium perborate. These tooth whitening agents liberate active oxygen and hydrogen peroxide. Tooth whitening agents can be present in tooth whitening compositions at a concentration of from about 0.1% to about 90%; typically, the concentration of carbamide peroxide in tooth whitening compositions is from about 10% to about 25%.

Many tooth whitening compositions are known in the art, including aqueous solutions, gels, pastes, liquids, films, strips, one-part systems, two-part systems, compositions that require activation of the tooth whitening agent (e.g., by inclusion of a radiant-energy or heat-energy absorbing substance, such as substantially conjugated hydrocarbons, which activates the bleaching agent when irradiated), etc. See, e.g., U.S. Pat. Nos. 5,302,375, 5,785,887, 5,858,332, 5,891,453, 5,922,307, 6,322,773, 6,419,906, and PCT applications WO 99/37236, WO 01/89463 and WO 02/07695, the complete disclosures of which are incorporated herein by reference. Also, many other oral care compositions (e.g., toothpastes) and devices (e.g., dental flosses) comprise a tooth whitening agent.

The use of tooth whitening compositions, or of one of the many oral care compositions and devices which comprise a tooth whitening agent, can cause inflammation of the tissues of the mouth. Incorporation of a compound of formula I or a pharmaceutically-acceptable salt thereof in tooth whitening compositions or other oral care compositions and devices comprising a tooth whitening agent will inhibit the inflammation. Alternatively, an oral care composition or device comprising a compound of formula I or a pharmaceutically-acceptable salt thereof can be used before or after the tooth whitening composition or oral care composition or device comprising a tooth whitening agent to inhibit the inflammation.

For instance, teeth are commonly whitened by applying a tooth whitening composition to the teeth by means of a dental tray or trough. A compound of formula I or a pharmaceutically-acceptable salt thereof could be incorporated into the tooth whitening composition that is used in the tray or trough. Alternatively, a separate composition comprising a compound of formula I or a pharmaceutically-acceptable salt thereof could be applied to the teeth in a cleaned or different tray or trough after the application of the tooth whitening composition is completed. In a further alternative, a wash or rinse comprising a compound of formula I or a pharmaceutically-acceptable salt thereof could be used to rinse the mouth before and/or after the application of the tooth whitening composition.

A recently developed product for applying a tooth whitening composition to the teeth is a flexible strip. See, e.g., U.S. Pat. Nos. 5,891,453 and 6,419,906. A compound of formula I or a pharmaceutically-acceptable salt thereof could be incorporated into such strips. For instance, a compound of formula I or a pharmaceutically-acceptable salt thereof could be incorporated into the tooth whitening composition, which is then applied to the strips, or a solution, gel or other composition comprising a compound of formula I or a pharmaceutically-acceptable salt thereof could be separately applied to the strips, either during their manufacture or just prior to use by the patient. In yet another alternative, strips comprising a tooth whitening composition and strips comprising a compound of formula I or a pharmaceutically-acceptable salt thereof could both be supplied to the patient and would be used sequentially.

The oral care compositions of the invention may comprise a single phase or a plurality of phases. A plurality of phases will be used, e.g., where some of the ingredients are incompatible, some of the ingredients are unstable, or the ingredients are best combined at the time of use. Thus, one of the phases will include some of the ingredients, and the remainder of the ingredients will be contained in one or more additional phases. The plurality of phases may be a plurality of separate compositions, in which case the plurality of phases will be provided in a plurality of separate containers or in a plurality of compartments in a single container, and the plurality of phases will be combined at the time of use. As an alternative, the plurality of phases may be formed by encapsulating some of the ingredients, in which case the plurality of phases may all be contained in a single container. Multi-phase oral care compositions are described in, e.g., U.S. Pat. Nos. 5,302,375, 5,906,811, 5,976,507, 6,228,347 and 6,350,438 and PCT application number WO 99/37236.

The invention also provides oral care devices comprising a compound of formula I or a pharmaceutically-acceptable salt thereof. Oral care devices of the invention include devices intended for use by consumers and patients and devices intended for use by dental professionals (e.g., dental hygienists, dentists and oral surgeons).

The oral care devices of the invention include surgical materials (such as sutures and sponges), flosses, tapes, chips, strips, fibers, a toothpick or rubber tip, dental implants and dental appliances (such as trays and troughs that fit over and cover the teeth and, optionally, the periodontal tissue) having a compound of formula I or a pharmaceutically-acceptable salt thereof adhered to, absorbed into, bound to, attached to, entrapped in, coated onto, or otherwise incorporated into, them. See, e.g., U.S. Pat. Nos. 5,709,873, 5,863,202, 5,891,453, 5,967,155, 5,972,366, 5,980,249, 6,026,829, 6,080,481, 6,102,050, 6,350,438, 6,419,906, PCT application WO 02/13775, and EP application 752833, which describe such oral care devices and methods of incorporating compounds into them (the complete disclosures of all of these patents and applications are incorporated herein by reference). For instance, a compound of formula I or a pharmaceutically-acceptable salt thereof can be incorporated into a binder (e.g., a wax or polymer) and coated onto dental floss, dental floss can be soaked in a bath of a liquid containing a compound of formula I or a pharmaceutically-acceptable salt thereof to impregnate or coat the floss with the compound(s), a compound of formula I or a pharmaceutically-acceptable salt thereof in solid (e.g., freeze-dried) form can be incorporated into a polymer film suitable for application to the teeth, a compound of formula I or a pharmaceutically-acceptable salt thereof in a solution or gel can be applied to a flexible strip suitable for application to teeth, or a suture or other surgical material can be soaked in a solution containing a compound of formula I or a pharmaceutically-acceptable salt thereof followed by removal of the solvent so that the compound(s) become associated with (bound to, entrapped in, coated onto, etc.) the suture or surgical material. See, e.g., U.S. Pat. Nos. 5,891,453, 5,967,155, 5,972,366, 6,026,829, 6,080,481, 6,102,050, and 6,419,906.

Also included within the scope of the invention are oral care products for animals, such as foods, chews, and toys. Suitable products are described in U.S. Pat. No. 6,350,438.

A compound of formula I or a pharmaceutically-acceptable salt thereof can be used to treat a tissue of an animal's mouth. “Mouth” is used herein to mean the cavity bounded externally by the lips and internally by the pharynx that encloses the tongue, gums and teeth. Thus, the tissues of the mouth include the lips, tongue, gums, buccal tissue, palate and teeth. A single tissue, a plurality of tissues, a portion of one or more tissues, all or substantially all of the tissues of the mouth, or combinations of the foregoing, may be treated according to the invention.

To treat a tissue of the mouth, the tissue is contacted with a compound of formula I or a pharmaceutically-acceptable salt thereof. For instance, the tissue may be contacted with an oral care composition comprising a compound of formula I or a pharmaceutically-acceptable salt thereof. Methods of contacting tissues of the mouth with oral care compositions are well known in the art. Suitable methods include rinsing the tissue with a solution (e.g., a mouthwash, rinse, spray, liquid dentrifice, or other solution), brushing the teeth with a dentrifice (e.g., a toothpaste, tooth gel, or powder), applying a non-abrasive solution, gel, paste, cream or ointment directly to the tissue (with or without the use of an applicator), chewing gum, chewing or sucking a lozenge, mint or tablet, and many other means of topical application. Suitable applicators for applying oral care compositions, such as solutions, gels, pastes, creams and ointments, to a tissue include a swab, a stick, a plastic paddle, a dropper, a syringe, a strip (such as those described in U.S. Pat. Nos. 5,891,453 and 6,419,906), a finger, or a dental tray or appliance (such as those shown in U.S. Pat. Nos. 5,863,202 and 5,980,249 and EP application 752833) which allows for immersion of the teeth and, optionally, the periodontal tissue in, e.g., a gel or solution. In addition, to treat a tissue of the mouth, the tissue may be contacted with an oral care device comprising a compound of formula I or a pharmaceutically-acceptable salt thereof. Methods of contacting tissues of the mouth with oral care devices are well known in the art. For instance, sutures can be used to close a surgical wound or a wound resulting from a tooth extraction, dental floss can be used to floss the teeth, etc.

The treatment of the tissue can be prophylactic treatment. For instance, the tissue may be treated as part of a prophylactic oral care regimen. A compound of formula I or a pharmaceutically-acceptable salt thereof can be incorporated into an oral care composition or device, such as a toothpaste, a tooth gel, a mouthwash or rinse, or a dental floss, that is employed in such a regimen and will be used regularly, preferably at least once per day, more preferably two or three times per day. In another alternative, a compound of formula I or a pharmaceutically-acceptable salt thereof may be contained in a separate oral care composition or device which will be used separately from other compositions and devices employed in the prophylactic oral care regimen. For instance, a compound of formula I or a pharmaceutically-acceptable salt thereof can be incorporated into a mouthwash or rinse, a gum, a lozenge or a chewable tablet, which would be used regularly, preferably at least once per day, more preferably at least two or three times per day.

Tissues may also be treated prophylactically in connection with a variety of dental procedures, including surgeries and tooth extractions. For instance, the tissue(s) on which surgery is being performed, those tissues near the area where the surgery is being performed or, for ease of treatment, all or substantially of the tissues of the mouth, can be treated prior to surgery, during surgery, after the surgery, or combinations thereof. Similarly for a tooth extraction, the tissue(s) surrounding the tooth which is to be extracted, adjacent tissues or, for ease of treatment, all or substantially of the tissues of the mouth, can be treated prior to tooth extraction, during the tooth extraction, after the tooth extraction, or combinations thereof. For instance, the mouth could be rinsed prior to surgery or tooth extraction with a solution comprising a compound of formula I or a pharmaceutically-acceptable salt thereof, the wound(s) caused by the surgery or tooth extraction could be closed with sutures having a compound of formula I or a pharmaceutically-acceptable salt thereof incorporated into them, and/or the mouth could be rinsed immediately after the surgery or tooth extraction, and/or at intervals thereafter, with a solution comprising a compound of formula I or a pharmaceutically-acceptable salt thereof. Finally, as described above, tissues may be treated prophylactically in connection with the whitening of the teeth of an animal.

A compound of formula I or a pharmaceutically-acceptable salt thereof can also be used to treat diseases and conditions of the mouth, such as inflammation and inflammatory diseases and conditions. Specific diseases and conditions treatable with a compound of formula I or a pharmaceutically-acceptable salt thereof include gingivitis, periodontitis, infections (bacterial infections, viral infections, yeast infections and fungal infections), ulcers, cold sores, canker sores and inflammation accompanying surgery or tooth extraction. The treatment of other diseases and conditions of the mouth, such as cancer, is more typically performed by, or under the supervision of, a medical doctor, rather than a dentist. Accordingly, the treatment of these disease and conditions was dealt with above in the discussion of therapeutic methods and pharmaceutical products. However, the use of the oral care products of the invention and the use of the pharmaceutical products of the invention together in the treatment of these types of diseases and conditions of the mouth should be beneficial.

It is understood by those skilled in the art that the dosage amount of a compound of formula I or a pharmaceutically-acceptable salt thereof needed to treat a tissue of an animal's mouth will vary depending on whether the treatment is prophylactic or for the treatment of a disease or condition, the identity of the disease or condition to be treated, the severity of the disease or condition, the type of oral care composition used, the duration of the treatment, the identify of any other drugs being administered to the animal, the age, size and species of the animal, and like factors known in the medical and veterinary arts. In general, a suitable daily dose of a compound of the present invention will be that amount of the compound which is the lowest dose effective to produce a therapeutic effect. It is expected that usage of oral care compositions comprising from about 0.000001% to about 20% of a compound of formula I or a pharmaceutically-acceptable salt thereof one or more times per day will provide effective daily dosages. However, the actual daily dosage to be employed, the number of treatments per day, and the length of treatment will be determined by an attending dentist or veterinarian within the scope of sound medical judgment.

The invention also provides a kit comprising an oral care product according to the invention. In the case where the oral care product is an oral care composition, the kit may also include an applicator for applying the oral care composition to a tissue of an animal's mouth, such as a swab, a stick, a plastic paddle, a dropper, a syringe, a strip (such as that described in U.S. Pat. Nos. 5,891,453 and 6,419,906) or a dental tray or appliance (such as those shown in U.S. Pat. Nos. 5,863,202 and 5,980,249 and EP application 752833) which allows for immersion of the teeth and, optionally, the periodontal tissue in, e.g., a gel or solution. The kit could also include a cup, vial or other device for dispensing and/or measuring the amount of the oral care composition of the invention needed for the intended use. Of course, the kits could include both an oral care composition and an oral care device according to the invention. In addition to an oral care composition and/or device of the invention, the kits could also comprise another type of oral care composition or device, such as a tooth whitening composition, strips comprising a tooth whitening agent, applicators for applying oral care compositions, etc. Kits according to the invention will also include instructions for using the kit and/or the oral care product of the invention and may include any other desired items.

E. Personal Care Products and Methods

A compound of formula I or a pharmaceutically-acceptable salt thereof can also be administered to an animal in personal care products. Personal care products include personal care compositions and personal care devices.

Personal care compositions and devices of the invention include compositions and devices intended for use by consumers and patients and compositions and devices intended for use by professionals (e.g., dermatologists, beauty salons and spas).

Personal care compositions include cosmetics, skin creams and lotions, face and body moisturizers, suntan creams and lotions, oils, washes, rinses, solutions, eye drops, emulsions, liquids, gels, ointments, sprays, powders, deodorants, shampoos, scalp treatment compositions, lip glosses, lip balms, anti-acne preparations, analgesics, etc.

The personal care compositions of the invention comprise a compound of formula I or a pharmaceutically-acceptable salt thereof and a pharmaceutically-acceptable carrier. The personal care compositions may also comprise one or more other acceptable ingredients, including other active compounds and/or other ingredients conventionally used in personal care compositions. Each carrier and ingredient must be “acceptable” in the sense of being compatible with the compound of formula I or pharmaceutically-acceptable salt thereof and any other ingredients of the composition and not being injurious to the animal. Suitable ingredients for use in personal care compositions and methods of making and using personal care compositions are well known in the art.

A wide variety of carriers suitable for use in skin care compositions are well known in the art. For example, emulsion carriers (including oil-in-water, water-in-oil, water-in-oil-in-water and oil-in-water-in-silicone emulsions) can be used. These emulsions can cover a broad range of viscosities (e.g., from about 100 centipoise (cps) to about 200,000 cps). Other suitable carriers include: anhydrous liquid solvents, such as oils, alcohols and silicones (e.g., mineral oil, ethanol, isopropanol, dimethicone, cyclomethicone and the like); aqueous-based single phase liquid solvents (e.g., hydro-alcoholic solvent systems); and thickened versions of these anhydrous and aqueous-based single phase solvents (e.g., where the viscosity of the solvent has been increased to form a solid or semi-solid by the addition of appropriate gums, resins, waxes, polymers, salts and the like). The carrier preferably comprises from about 50% to about 99% by weight of the skin care compositions, more preferably from about 75% to about 99%, most preferably from about 85% to about 95%.

A wide variety of carriers suitable for use in hair care compositions are also well known in the art. For instance, water, alcohols (e.g., methanol, ethanol and isopropanol) and mixtures thereof can be used. The carriers can also comprise a wide variety of additional materials including acetone, hydrocarbons (e.g., isobutane, hexane, decene), linalool, esters (e.g., ethyl acetate and dibutyl phthalate), volatile silicone derivatives (e.g., siloxanes, such as phenyl pentamethyl disiloxane, methoxypropyl heptamethyl cyclotetrasiloxane, chloropropyl pentamethyl disiloxane, hydroypropyl pentamethyl disiloxane, octamethyl cyclotetrasiloxane, decamethyl cyclopentasiloxane, cyclomethicone and dimethicone), and mixtures thereof. Hair care products having a low viscosity may also utilize an emulsifying agent (preferably at a level of from about 0.01% to about 7.5% by weight of the composition). The carrier will comprise from about 0.5% to about 99.5% by weight of the hair care compositions, preferably from about 5.0% to about 99.5%, more preferably from about 10.0% to about 98.0%.

In addition to a compound of formula I or a pharmaceutically-acceptable salt thereof and the carrier, the personal care compositions of the invention can comprise a wide variety of additional ingredients. These additional ingredients include pharmaceutically active ingredients (e.g., anti-acne actives, analgesic actives, antipruritic actives, anesthetic actives and antimicrobial actives), other active ingredients (e.g., sunscreening actives, sunless tanning actives, skin bleaching actives, anti-dandruff actives, antiperspirant actives and deodorant actives), conditioners, humectants, moisturizers, surfactants, thickeners, emollients and other ingredients commonly used in personal care compositions.

As noted above, the pharmaceutically active ingredients that can be included in the personal care compositions of the invention in addition to a compound of formula I or a pharmaceutically-acceptable salt thereof include anti-acne actives, analgesic actives, antipruritic actives, anesthetic actives and antimicrobial actives. Amounts of these ingredients to include in the compositions are known in the art or can be determined empirically. Suitable dosage amounts will vary with, e.g., the specific active ingredient, the ability of the compositions to penetrate the active through the skin, the amount of composition to be applied, the particular condition being treated, the age and physical condition of the animal being treated, the severity of the condition, the duration of the treatment, the nature of concurrent therapy and like factors.

Anti-acne actives include the keratolytics (such as salicylic acid, sulfur, lactic acid, glycolic, pyruvic acid, urea, resorcinol and N-acetylcysteine), retinoids (such as retinoic acid and its derviatives), antibiotics and antimicrobials (such as benzoyl peroxide, octopirox, erythromycin, zinc, tetracycline, triclosan, azelaic acid and its derivaties, phenoxy ethanol, phenoxy propanol, ethylacetate, clindamycin and meclocycline), sebostats (such as flavinoids), alpha and beta hydroxy acids, and bile salts (such as scymnol sulfate and its derivatives, deoxycholate and cholate).

Analgesic actives include salicylic acid derivatives (such as methyl salicylate), species and derivatives of the genus capsicum (such as capsaicin), steroids (such as hydrocortisone) and non-steroidal anti-inflammatory drugs (NSAIDS). The NSAIDS can be selected from the following categories: propionic acid derivatives (aspirin, acetaminophen, ibuprofen, naproxen, benoxaprofen, flurbiprofen, fenoprofen, fenbufen, ketoprofen, indoprofen, pirprofen, carprofen, oxaprozin, pranoprofen, miroprofen, tioxaprofen, suprofen, alminoprofen, tiaprofenic acid, fluprofen and bucloxic acid), acetic acid derivatives, fenamic acid derivatives, biphenylcarboxylic acid derivatives and oxicams.

Antipruritic actives include the pharmaceutically-acceptable salts of methdilizine and trimeprazine.

Anesthetic actives include the pharmaceutically-acceptable salts of lidocaine, bupivacaine, chlorprocaine, dibucaine, etidocaine, mepivacaine, tetracaine, dyclonine, hexylcaine, procaine, cocaine, ketamine, pramoxine and phenol.

Antimicrobial (antibacterial, antifungal, antiprotozoal and antiviral) actives include pharmaceutically-acceptable salts of β-lactams, quinolones, ciprofloxacin, norfloxacin, tetracycline, erythromycin, amikacin, triclosan, doxycycline, capreomycin, chlorhexidine, chlortetracycline, oxytetracycline, clindamycin, ethambutol, metronidazole, pentamidine, gentamicin, kanamycin, lineomycin, methacycline, methenamine, minocycline, neomycin, netilmicin, paromomycin, streptomycin, tobramycin, miconazole, amanfadine, octopirox, parachlorometa xylenol, nystatin, tolnaftate and clotrimazole.

Sunscreening agents include 2-ethylhexyl p-methoxycinnamate, 2-ethylhexyl N,N-dimethyl-p-aminobenzoate, p-aminobenzoic acid, 2-phenylbenzimidazole-5-sulfonic acid, octocrylene, oxybenzone, homomethyl salicylate, octyl salicylate, 4,4′-methoxy-t-butyldibenzoylmethane, 4-isopropyl dibenzoylmethane, 3-benzylidene camphor, 3-(4-methylbenzylidene) camphor, titanium dioxide, zine oxide, silica, iron oxide, and mixtures thereof. Additional sunscreening agents include those having, in a single molecule, two distinct chromophore moities which exhibit different ultraviolet radiation absorption spectra (one absorbs predominantly in the UVA range and one absorbs predominantly in the UVB range), such as 4-N,N-(2-ethylhexyl)methylaminobenzoic acid ester of 2,4-dihydroxybenzophenone, 4-N,N-(2-ethylhexyl)methylaminobenzoic acid ester of 4-hydroxydibenzoylmethane, 4-N,N-(2-ethylhexyl)methylaminobenzoic acid ester of 2-hydroxy-4-(2-hydroxyethoxy)benzophenone, 4-N,N-(2-ethylhexyl)methylaminobenzoic acid ester of 4-(2-hydroxyethoxy)dibenzoylmethane, and mixtures thereof. See also PCT application WO 03/013468 which describes additional suitable sunscreening agents. Generally, the sunscreens will comprise from about 0.5% to about 20% by weight of the compositions. Exact amounts will vary depending upon the sunscreen chosen and the desired Sun Protection Factor (SPF). SPF is a commonly used measure of photoprotection of a sunscreen against erythema. See Federal Register, Volume 43, No. 166, pages 38206-38269, Aug. 25, 1978.

Sunless tanning actives include dihydroxyacetone, glyceraldehyde, indoles and their derivatives, and the like.

Skin bleaching actives include hydroquinone, ascorbic acid, kojic acid and sodium metabisulfite.

Anti-dandruff actives include zinc pyrithione, octopirox, selenium disulfide, sulfur, coal tar and the like.

Antiperspirant actives include astringent metallic salts, such as the inorganic and organic salts of aluminum, zirconium and zinc, as well as mixtures thereof.

Deodorant actives include bacteriostats (e.g., 2,2′-methylenebis(3,4,6-trichlorophenol), 2,4,4′-trichloro-2′-hydroxy(diphenyl ether) (also known as triclosan), zinc phenolsulfonate, 2,2′-thiobis(4,6-dichlorophenol), p-chloro-m-xylenol, dichloro-m-xylenol, sodium N-lauroyl sarcosine, sodium N-palmitoyl sarcosine, lauroyl sarcosine, N-myristoyl glycine, potassium N-lauroyl sarcosine, aluminum chlorhydroxy lactate, and the like).

Conditioning agents useful in the compositions, especially the hair care compositions, include hydrocarbons, silicone fluids and cationic materials. The hydrocarbons can be either straight or branched-chain and can contain from about 10 to about 16 carbon atoms. Examples of suitable hydrocarbons include decane, dodecane, tetradecane, tridecane and mixtures thereof. Silicone conditioning agents include cyclic or linear polydimethylsiloxanes, phenyl and alkyl phenyl silicones, and silicone copolyols. Cationic conditioning agents include quaternary ammonium salts (e.g., dialkyl dimethyl ammonium salts wherein the alkyl groups have 12-22 carbon atoms (such as ditallow dimethyl ammonium chloride, ditallow dimethyl ammonium methyl sulfate, dihexadecyl dimethyl ammonium chloride and di(hydrogenated tallow) ammonium chloride) and dicationics (such as tallow propane diammonium dichloride)), quaternary imidazolinium salts (e.g., imidazolinium salts containing alkyl groups containing 12-22 carbon atoms (such as 1-methyl-1[(stearoylamide)ethyl]-2-heptadecyl-4,5-dihydroimidazolinium chloride, 1-methyl-1[(palmitoylamide)ethyl]-2-octadecyl-4,5-dihydroimidazolinium chloride and 1-methyl-1[(tallowamide)ethyl]-2-tallow-imidazolinium methyl sulfate)) and the salts of fatty amines (e.g., stearylamine hydrochloride, soyamine hydrochloride and stearylamine formate).

Humectants and moisturizing agents include urea, guanidine, glycolic acid and glycolate salts (e.g., ammonium and quaternary alkyl ammonium), lactic acid and lactate salts (e.g, ammonium and quaternary alkyl ammonium), aloe vera in any of its variety of forms (e.g., aloe vera gel), polyhydroxy alcohols (e.g, sorbitol, glycerol, hexanetriol, propylene glycol, butylene glycol, hexylene glycol and the like)), polyethylene glycols, sugars and starches, sugar and starch derivatives (e.g., alkoxylated glucose), hyaluronic acid, lactamide monoethanolamine, acetamide monoethanolamine, and mixtures thereof. These agents will generally be present at a level of from about 0.1% to about 20% of the weight of the compositions.

Surfactants useful in the compositions include anionic, nonionic, cationic, zwitterionic and amphoteric surfactants. Suitable anionic surfactants include long chain sulfates, sulfonates, isethionates, carboxylates, taurates, and sulfosuccinates, such as alkyl glyceryl ether sulfonate, ammonium lauryl sulfate, ammonium laureth sulfate, triethylamine lauryl sulfate, triethylamine laureth sulfate, triethanolamine lauryl sulfate, triethanolamine laureth sulfate, monoethanolamine lauryl sulfate, monoethanolamine laureth sulfate, diethanolamine lauryl sulfate, diethanolamine laureth sulfate, lauric monoglyceride sodium sulfate, sodium lauryl sulfate, sodium laureth sulfate, potassium lauryl sulfate, potassium laureth sulfate, sodium lauryl sarcosinate, sodium lauroyl sarcosinsate, lauryl sarcosine, cocoyl sarcosine, ammonium cocoyl sulfate, ammonium lauroyl sulfate, sodium cocoyl sulfate, sodium lauroyl suflate, potassium cocoyl sulfate, potassium lauryl sulfate, triethanolamine lauryl sulfate, monoethanolamine cocoyl sulfate, monoethanolamine lauryl sulfate, sodium tridecyl benzene sulfonate and sodium dodecyl benzene sulfonate. For cationic surfactants, see U.S. Pat. No. 5,916,548 and the references cited therein. Nonionic surfactants include the compounds produced by condensation of alkylene oxide groups (hydrophilic in nature) with an organic hydrophobic compound, which may be aliphatic or alkyl aromatic in nature. Amphoteric and zwitterionic surfactants include betaines, such as amidocarboxybetaines, alkyl betaines, amidopropyl betaines, amidopropyl sultaines and sulfobetaines. Additional amphoteric and zwitterionic surfactants include derivatives of aliphatic quaternary ammonium and sulfonium compounds, in which the aliphatic radicals can be straight or branched chain and wherein one of the aliphatic substituents contains from about 8-18 carbon atoms and one contains an anionic water-solubilizing group (e.g., carboxy, sulfonate or sulfate). Further amphoteric and zwitterionic surfactants include derivatives of aliphatic secondary and tertiary amines, in which the aliphatic radicals can be straight or branched chain and wherein one of the aliphatic substituents contains from about 8-18 carbon atoms and one contains an anionic water-solubilizing group (e.g., carboxy, sulfonate or sulfate), such as sodium 3-dodecyl-aminopropionate, sodium 3-dodecylamino propane sulfonate and N-alkyl taurines. The surfactant or mixture of surfactants will generally be present at a level of from about 0.2% to about 30% of the weight of the compositions.

Thickeners include carboxylic acid polymers (described in U.S. Pat. No. 5,916,548, the complete disclosure of which is incorporated herein by reference). These crosslinked polymers contain one or more monomers derived from acrylic acid, substituted acrylic acids and salts and esters of these acrylic acids and the substituted acrylic acids, wherein the crosslinking agent contains two or more carbon-carbon double bonds and is derived from a polyhydric alcohol. Specific examples of such polymers are the carbomers, which are homopolymers of acrylic acid crosslinked with allyl ethers of sucrose or pentaerytritol (available as the Carbopolt 900 series from B.F. Goodrich), and copolymers of C₁₀₋₃₀ alkyl acrylates with one or more monomers of acrylic acid, methacrylic acid or one of their short chain (C₁₋₄ alcohol) esters, wherein the crosslinking agent is an allyl ether of sucrose or pentaerytritol (also known as acrylates/C10-30 alkyl acrylate crosspolymers and available as Carbopolt 1342, Pemulen TR-1 and Pemulen TR-2 from B.F. Goodrich). Other thickeners include xanthan gum, guar gum, carboxymethyl cellulose, hydroxymethyl cellulose, hydroxyethyl cellulose, alkyl modified hydroxyalkyl celluloses (e.g, long chain alkyl modified hydroxyethyl celluloses, such as cetyl hydroxyethyl cellulose) and magnesium aluminum silicate. These thickeners will generally be present at a level of from about 0.025% to about 1% of the weight of the compositions.

Emulsifiers suitable for use in personal care compositions can be any of a wide variety of nonionic, cationic, anionic and zwitterionic emulsifiers. Examples of suitable emulsifiers include esters of glycerin, esters of propylene glycol, fatty acid esters of polyethylene glycol, fatty acid esters of polypropylene glycol, esters of sorbitol, esters of sorbitan anhydrides, carboxylic acid copolymers, esters and ethers of glucose, ethoxylated ethers, ethoxylated alcohols, fatty acid amides, acyl lactylates, soaps, and mixtures thereof. Specific suitable emulsifiers include polyethylene glycol 20 sorbitan monolaurate (Polysorbate 20), polyethylene glycol 5 soya sterol, Steareth-20, Ceteareth-20, PPG-2 methyl glucose ether distearate, Ceteth-10, Polysorbate 80, Polysorbate 60, glyceryl stearate, PEG-100 stearate and mixtures thereof. The emulsifiers will generally be present at a level of from about 0.1% to about 10% of the weight of the compositions.

Emollients include volatile and nonvolatile silicone oils, highly branched hydrocarbons and nonpolar carboxylic acid and alcohol esters, and mixtures thereof. The emollients will generally be present at a level of from about 1% to about 50% of the weight of the compositions.

A variety of additional ingredients can be included in the personal care compositions. These additional ingredients include vitamins and derivatives thereof (e.g., ascorbic acid, vitamin E tocopheryl acetate, retinoic acid, retinol, retinoids and the like), pH adjusting agents (see discussion above in description of oral care products), polyquatemium and mineral oil, resins, gums, polymers for aiding in the film-forming properties and substantivity of the composition (such as a copolymer of eicosene and vinyl pyrrolidone), suspending agents (e.g., ethylene glycol distearate and the like), preservatives, skin penetration aids, antioxidants, chelators, sequestrants and aesthetic components (e.g., fragrances, colorings, essential oils, skin sensates, astringents and skin soothing agents; specific examples of such aesthetic components include panthenol and its derivatives, pantothenic acid and its derivatives, clove oil, menthol, camphor, eucalyptus oil, eugenol, menthyl lactate, witch hazel distillate, allantoin and bisabalol).

It will be appreciated that a wide variety of different personal care compositions can be prepared utilizing the above described ingredients and other ingredients known in the art or which will be developed. It is within the skill in the art to choose appropriate ingredients and combinations of ingredients and to determine an effective amount of a compound of formula I or a pharmaceutically-acceptable salt thereof to include in a particular personal care composition.

The invention also provides personal care devices. Personal care devices include surgical materials (such as sutures and sponges), bandages, sponges, cloths, swabs, pads and wipes. The personal care devices of the invention will have a compound of formula I or a pharmaceutically-acceptable salt thereof adhered to, absorbed into, adsorbed onto, bound to, attached to, entrapped in, impregnated in, coated onto or otherwise incorporated into, them. For instance, a device can be soaked in a solution of a compound of formula I or a pharmaceutically-acceptable salt thereof, followed by removal of the solvent, to adhere, absorb, adsorb, bind, attach, entrap, impregnate, coat the device with the compound of formula I or pharmaceutically-acceptable salt thereof. See, e.g., the description above of the preparation of oral care devices.

The invention also provides a method for the care and treatment of the skin. The method comprises contacting an animal's skin with an effective amount of a compound of formula I or a pharmaceutically-acceptable salt thereof. For instance, the skin may be contacted with a personal care composition comprising a compound of formula I or a pharmaceutically-acceptable salt thereof. Methods of contacting the skin with personal care compositions are well known in the art. Suitable methods include washing the skin with a cleaning solution, rinsing the skin with a rinse, applying a solution, gel, cream, lotion or ointment on the skin (with or without the use of an applicator), washing the hair with a shampoo that contacts the scalp, and many other means of topical application. Suitable applicators for applying personal care compositions include a cotton ball, a gauze pad, a wipe, a cloth, a swab, a dropper, a syringe or a finger. In addition, the skin may be contacted with a personal care device comprising a compound of formula I or a pharmaceutically-acceptable salt thereof. Methods of contacting the skin with personal care devices are well known in the art. For instance, sutures can be used to close a surgical wound, a wipe or pad impregnated with a compound of formula I or a pharmaceutically-acceptable salt thereof can be used to clean the skin, a bandage comprising the compound of formula I or pharmaceutically-acceptable salt thereof can be applied to the skin, etc.

The treatment of the skin can be prophylactic treatment. For instance, the skin may be treated as part of a prophylactic skin care regimen. A compound of formula I or a pharmaceutically-acceptable salt thereof can be incorporated into a personal care composition or device that is employed in such a regimen or the compound of formula I or pharmaceutically-acceptable salt thereof may be contained in a separate personal care composition or device which will be used separately from other compositions and devices employed in the prophylactic skin care regimen. The prophylactic regimen is performed regularly (e.g., monthly or daily).

Skin may also be treated prophylactically in connection with a variety of dermatological procedures, including surgeries, dermabrasions and chemical peels. For instance, the area of skin on which surgery is to be performed can be treated prior to surgery, during surgery, after the surgery, or combinations thereof. For instance, the skin could be rinsed prior to surgery with a solution comprising a compound of formula I or a pharmaceutically-acceptable salt thereof, the wound(s) caused by the surgery could be closed with sutures having a compound of formula I or a pharmaceutically-acceptable salt thereof incorporated into them, and/or the skin could be rinsed immediately after the surgery, and/or at intervals thereafter, with a solution comprising a compound of formula I or a pharmaceutically-acceptable salt thereof.

A personal care product comprising a compound of formula I or a pharmaceutically-acceptable salt thereof can also be used to treat a disease or condition of the skin. Specific diseases and conditions treatable according to the invention are described above in the discussion of therapeutic methods and pharmaceutical compositions. It will be appreciated that diseases and conditions of the skin can be treated with a pharmaceutical composition and/or a personal care composition or device.

It is understood by those skilled in the art that the dosage amount of a compound of formula I or a pharmaceutically-acceptable salt thereof needed to treat an animal's skin using a personal care product will vary depending on whether the treatment is prophylactic or for the treatment of a disease or condition, the identity of the disease or condition to be treated, the severity of the disease or condition, the type of personal care composition or device used, the duration of the treatment, the identify of any other drugs being administered to the animal, the age, size and species of the animal, and like factors.

The invention also provides a kit comprising a personal care product according to the invention. In the case where the personal care product is a personal care composition, the kit may also include an applicator for applying the personal care composition, such as a swab, cotton balls, wipes, pads, a plastic paddle, a squeeze bottle, a pump bottle, a dropper, or a syringe. The kit could also include a cup, vial or other device for dispensing and/or measuring the amount of the personal care composition of the invention needed for the intended use. Of course, the kits could include both a personal care composition and a personal care device according to the invention. In addition to a personal care composition and/or device of the invention, the kits could also comprise another type of personal care composition or device. Kits according to the invention will also include instructions for using the kit and/or the personal care product of the invention and may include any other desired items.

### EXAMPLE

### Example 1

Although N-acetyl-L-aspartate (NAA) has been shown to be important to myelin synthesis and osmotic regulation, the biological rationale for the high levels of NAA in the brain remains unknown. In this example, a human astroglial cell line (STTG) was treated with NAA and stimulated with either ionomycin or IL-1β. The subsequent inflammatory response was studied by measuring mediators of inflammation such as prostaglandin E₂ (PGE₂), cyclooxygenase-2 (COX-2) protein, and activated NFκB. PGE₂ levels in ionomycin-stimulated STTG cells decreased by 76% and >95% at NAA concentrations of 10 and 20 mM, respectively. Glutamate receptor antagonists (L-AP-4 and L-glutamic acid diethyl ester) also caused a decrease in PGE₂ levels in the STTG cell line. NAA also decreased the amounts of COX-2 protein and activated NFκB in IL-1β-stimulated STTG cells but had little effect on unstimulated cells. NAA had no effect on total COX-2 activity or COX-2 mRNA. These results demonstrate that NAA appears to be important in the modulation of inflammation in the human STTG astroglial cell line. Potential mechanisms for the anti-inflammatory action of NAA could be a result of acetylation of key pro-inflammatory enzymes (i.e., COX-2, IκBα kinase, etc.), glutamate receptor antagonism, or calcium chelation. The results of these findings are discussed in relation to neuronal pathologies that exhibit abnormal NAA levels within the brain.

A. Introduction

NAA is the second most abundant free amino acid in the mammalian brain next to glutamate (Tsai et al., Prog. Neurobiol., 46:531-540 (1995); Baslow, J. Neurochem., 68:1335-1344 (1997); Clark, Dev. Neurosci., 20:271-276 (1998)). NAA is located primarily in neurons at concentrations of approximately 10 mM (Jacobs et al., Magn. Reson. Med., 46:699-705 (2001); Wang et al., Magn. Reson. Med., 39:28-33 (1998); Pouwels et al., NMR Biomed., 10:73-78 (1997); Soher et al., Magn. Reson. Med., 35:356-363 (1996); Friedman et al., AJNR Am. J. Neuroradiol., 19:1879-1885 (1998)). Perturbations in NAA levels in the brain due to various pathological conditions have been detected with proton NMR spectroscopy. An elevation in NAA concentrations has been measured in Canavan's disease due to a lack of aspartoacylase (acylase II), the enzyme responsible for the breakdown of NAA (Baslow, Neurochem. Res., 28:941-953 (2003)). Decreases in NAA concentrations have been found in Huntington's disease (Jenkins et al., J. Neurochem., 74:2108-2119 (2000)), amyotrophic lateral sclerosis (Suhy et al., Neurology, 58:773-779 (2002)), Alzheimer's disease (Huang et al., Neurology, 57:626-632 (2001)), traumatic brain injury (Friedman et al., AJNR Am. J. Neuroradiol., 19:1879-1885 (1998)), ischemic injuries (Konaka et al., J. Cereb. Blood Flow Metab., 23:700-708 (2003)), multiple sclerosis (Wylezinska et al., Neurology, 60:1949-1954 (2003)), HIV (Iranzo et al., J. Neurol. Neurosurg. Psychiatry, 66:520-523 (1999)), and schizophrenia (Yamasue et al., Neuroreport, 13:2133-2137 (2002)).

In the brain, NAA is synthesized and stored in the neurons but is hydrolyzed in glial cells (Baslow, Neurochem. Res., 28:941-953 (2003)). In the brain interstitial space, the concentration of NAA is a 100-fold less than in the neuron (Sager et al., J. Neurochem., 68:675-682 (1997)). Therefore, a large efflux of NAA from neurons to the interstitial space is realized. Glial cells, by a specific transport mechanism, uptake this released NAA from neurons (Sager et al., J. Neurochem., 73:807-811 (1999); Huang et al., J. Pharmacol. Exp. Ther., 295:392-403 (2000)). In the glial cell, NAA is broken down into acetate and aspartate by aspartoacylase. Acetate is then utilized as an acetyl donor for synthesis of myelin lipids (Chakraborty et al., J. Neurochem., 78:736-745 (2001)). NAA has also been implicated in a neuronal molecular water pump that cotransports water and NAA extracellularly (Baslow, Neurochem. Int., 40:295-300 (2002)).

Although NAA has been shown to be important osmotically and for myelin synthesis, limited knowledge still exists about the purpose of the large amount of NAA present in the mammalian brain. In the present study, the inflammatory response of STTG astroglial cells and the effect that NAA had on this response were examined. Primarily, the result of NAA on cyclooxygenase-2 levels and prostaglandin synthesis was studied. The effect of NAA on the production of activated NFκB in stimulated STTG cells was also explored. These results could explain some of the pathologies associated with multiple brain disorders such as Canavan's disease.

B. Materials and Methods

Prostaglandin E₂ Assay. Human STTG astroglial cells (CRL-1718, American Type Tissue Collection, Rockville, Mass.) were grown in 75 cm² flasks (10% CO₂, 37° C.) containing AGM medium (BioWhittaker, Walkersville, Md.) to near confluency (>90%) prior to subculturing with trypsin/EDTA. Cells were plated on a 24-well plate at 50,000 cells/well and incubated for two days to near confluency prior to dosing with inhibitors. N-Acetyl aspartate (NAA, Sigma-Aldrich, St. Louis, Mo.) was dissolved in AGM medium and brought back to a pH of 7.4. Stock solutions of aspirin were dissolved in DMSO and then diluted in culture medium. DMSO concentrations were never >0.5% in the medium bathing the cells. Cells were then dosed with potential prostaglandin E₂ (PGE₂) inhibitors (NAA, L-2-Amino-4-phosphonobutyric acid [L-AP-4, Sigma-Aldrich, St. Louis, Mo.], L-glutamic acid diethyl ester [GDE, generous gift from Nagaraja K. R. Rao, DMI Synthesis UK Ltd.], aspirin, or dexamethasone). After a 1-hour incubation, the cells were then stimulated with 1 μM ionomycin (Sigma-Aldrich, St. Louis, Mo.) for 24 hours at 37° C. Total volume per well was 1 mL. Then, 0.5 mL of culture medium was removed from each well and immediately assayed for PGE₂ using the Prostaglandin E₂ enzyme immunoassay (EIA) system (Amersham, Piscataway, N.J.). The remaining medium containing the cells was assayed for cell viability using cell titer reagent (G358A, Promega, Madison, Wis.).

COX-2 Activity Assay. Modifying the method of Ouellet and co-workers (Proc. Natl. Acad. Sci. USA, 98:14583-14588 (2001)), COX-2 activity was determined using the PGE₂ EIA system. Briefly, COX-2 (Oxford Biomedical, Oxford, Mich.) was incubated at 37° C. for 5 minutes with arachidonic acid (AA), 500 μM phenol, and 1 μM hematin in a 100 mM phosphate buffer (pH 6.5). The reaction was stopped with a solution containing dH₂O/MeOH/1 M citric acid (30:4:1). After optimization of COX-2 and AA, 0.05 units of COX-2 per incubation and 1 μM of AA were determined to be optimal. Varying concentrations of NAA and aspirin (pH 6.5) were pre-incubated with COX-2, hematin, and phenol for 15 minutes at 37° C. prior to AA addition. The reaction was stopped after 5-minute incubation with AA. PGE₂ levels were determined using PGE₂ EIA system as described.

RT-PCR of COX-2. STTG cells were grown to near confluency on 25 cm² cell culture flasks. Cells were dosed with 5 or 10 mM NAA and incubated for 4 or 24 hours at 37° C., 10% CO₂. RNA was isolated using Rneasy Mini Kit (Qiagen, Valencia, Calif.) and quantitated using A₂₆₀/A₂₈₀ assay. Then, 2 μg of RNA was converted to cDNA using the Omniscript Reverse Transcriptase Kit (Qiagen, Valencia, Calif.). PCR was next performed using HotStarTaq Master Mix (Qiagen, Valencia, Calif.). COX-2 forward (TCTTTTAATGAGTACCGCAAACG [SEQ ID NO:1]) and reverse primers (TTAGACTTCTACAGTTCAGTCGAACG [SEQ ID NO:2]) were obtained from Qiagen (Valencia, Calif.) and used at a concentration of 0.5 μM. The PCR thermal cycler program consisted of: 1) 1 denaturation cycle of 15 minutes at 95° C.; 2) 30 cycles of 94° C. (30 sec denaturation), 55° C. (30 sec annealing), and 72° C. (30 sec elongation); and 3) 1 elongation cycle of 10 minutes at 72° C. PCR product was then loaded on a 2% (w/v) agarose gel and stained with ethidium bromide.

Western Blot of COX-2. STTG cells were treated with either NAA or aspirin and immediately stimulated with 1 or 2 ng/ml IL-1β (Sigma-Aldrich, St. Louis, Mo.). After a 24-hour incubation at 37° C. (10% CO₂), cells were lysed and total protein was quantitated by BCA (Pierce, Rockford, Ill.). Then, 0.5 mg lysate was immunoprecipitated using 1:500 goat anti-human COX-2 (Santa Cruz Biotechnology, Santa Cruz, Calif.) and Protein AG beads (Pierce, Rockford, Ill.) overnight at 4° C. Samples were loaded on a 4-20% Tris-Glycine gel (Invitrogen, Carlsbad, Calif.) and transferred to a nitrocellulose membrane overnight. The membrane was blocked with 5% Blotto (Santa Cruz Biotechnology, Santa Cruz, Calif.), and COX-2 was detected using 1:100 goat anti-human COX-2 (Santa Cruz Biotechnology, Santa Cruz, Calif.) and 1:5,000 rabbit anti-goat IgG HRP (Santa Cruz Biotechnology, Santa Cruz, Calif.). The membrane was visualized using ECL (Amersham, Piscataway, N.J.) and exposed to X-ray film.

NFκB Assay. STTG cells were treated with NAA and stimulated immediately with 1 ng/ml IL-1β. After a 24-hour incubation, cells were lysed and total protein was quantitated using the BCA™ Protein Assay Kit (Pierce, Rockford, Ill.). Then, 10 μg of protein lysate was assayed for NFκB using the TransAM™ NFκB Family Transcription Factor Assay Kit (Catalog No. 43296, Active Motif, Carlsbad, Calif.).

Statistical Methods. Statistical analysis was performed using t test analysis. All values are reported as mean ±SD.

C. Results

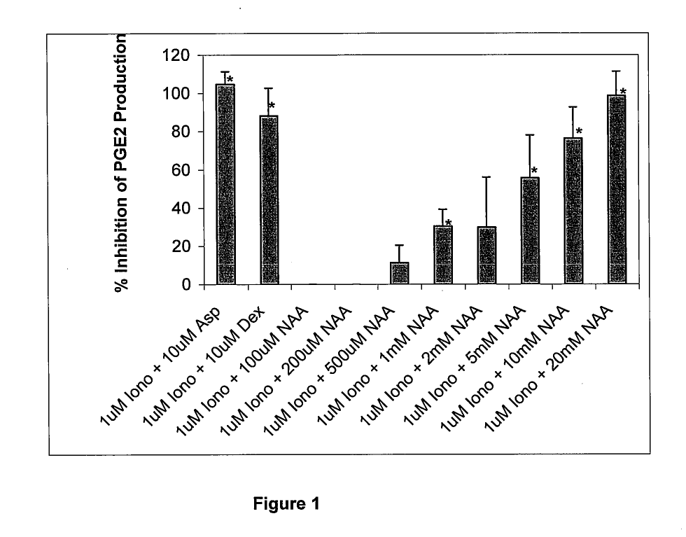

Effect of NAA on PGE₂ production. N-acetyl aspartate (NAA) was compared to aspirin (COX-1 and COX-2 inhibitor) and dexamethasone (COX-2 inhibitor only) in their ability to inhibit cyclooxygenases by measuring PGE₂ formation. STTG cells were dosed with the inhibitors prior to stimulation with ionomycin. Aspirin and dexamethasone demonstrated a complete inhibition of cyclooxygenases as evidenced by the lack of PGE₂ production in stimulated STTG cells (FIG. 1). NAA showed a dose-response decrease in PGE₂ production. At 5 mM NAA, there was a 56% decrease in PGE₂ production. At normal neuronal NAA concentrations (10 mM), a 76% decrease in PGE₂ production was measured, and at 20 mM, PGE₂ production was completely shutdown (>95% inhibition).

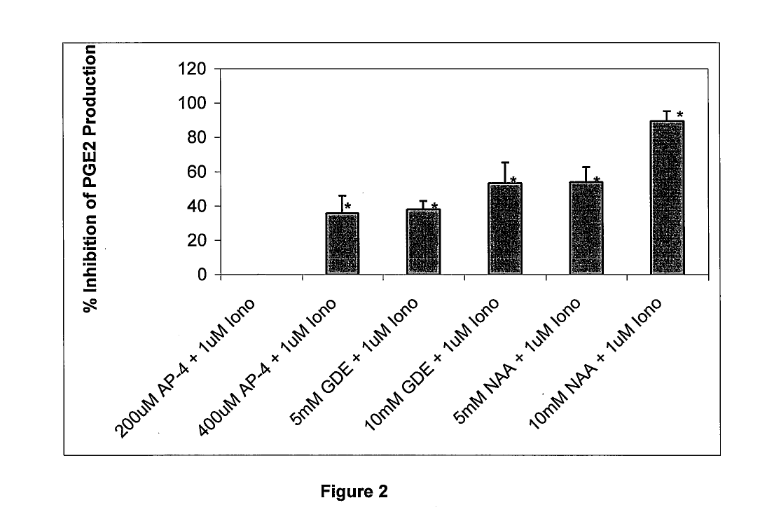

Effect of glutamate receptor antagonists. Known glutamate receptor antagonists, such as L-AP-4 and L-glutamic acid diethyl ester (GDE), did decrease total PGE₂ release in STTG cells stimulated with ionomycin as shown in FIG. 2. At equimolar concentrations, the decrease in PGE₂ release in the presence of GDE was not as dramatic as that caused by NAA at equimolar concentrations. At 5 and 10 mM GDE, there was a 40% and 60% decrease in PGE₂ release from STTG cells, respectively. A concentration of 5 mM NAA caused a 60% decrease in PGE₂ release, while a 90% decrease was seen in the presence of 10 mM NAA.

Effect of NAA on COX-2 activity and mRNA. To determine if NAA was inhibiting the cyclooxygenase enzyme directly, a COX-2 activity assay was developed using the PGE₂ EIA system. After optimizing for COX-2 and arachidonic acid (AA), a concentration of 0.05 units/incubation of COX-2 and 1 μM of AA was determined to be optimal. Applying these concentrations to incubations containing NAA, it was found that NAA, at concentrations up to 20 mM, had no effect on COX-2 activity (data not shown).

Total COX-2 mRNA in the presence of NAA was measured using RT-PCR. Unstimulated STTG astroglial cells produced a significant amount of signal that was not increased by ionomycin. NAA, at concentrations of 5 and 10 mM, did not decrease total COX-2 mRNA (data not shown).

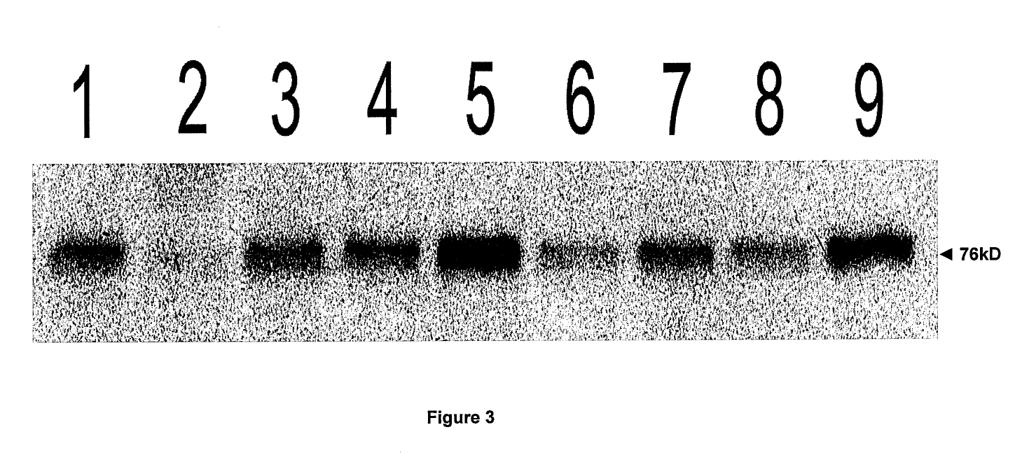

Effect of NAA on total COX-2 protein. COX-2 protein was quantitated by Western Blot techniques in STTG cells treated with NAA and stimulated with IL-1. FIG. 3 shows that aspirin had a decreasing effect on resting COX-2 protein in STTG cells, while NAA had no effect. On stimulated STTG cells, both aspirin and NAA decreased the total amount of COX-2 protein.

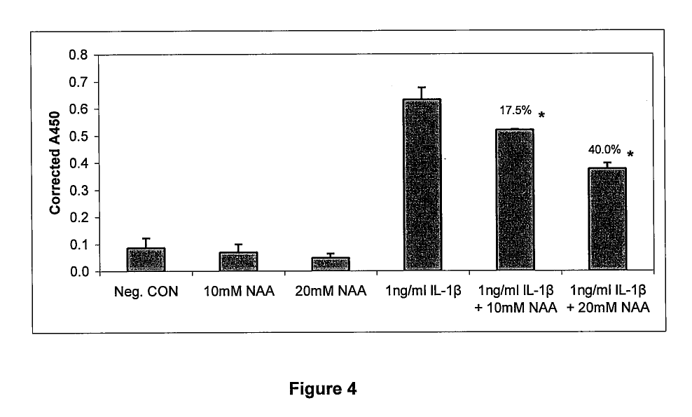

Effect of NAA on NFκB activation. Activated NFκB was quantitated using a 96-well plate coated with an oligonucleotide that contains an NFκB consensus-binding site. This site specifically binds activated NFκB contained in cell extracts. After stimulation with 1 ng/ml IL-1β, STTG cells showed a 7-fold increase in NFκB activation (FIG. 4). At concentrations of 10 mM and 20 mM, NAA decreased NFκB production in stimulated STTG cells by 17.5% and 40%, respectively.

D. Discussion

The cyclooxygenase (COX) subfamily of enzymes regulates the production of prostaglandins, including PGE₂. Two major COX isoforms have been described: COX-1, a housekeeping enzyme, and COX-2, an inducible enzyme upregulated in response to inflammation and trauma (Tegeder et al., FASEB J, 15:2057-2072 (2001)). Cyclooxygenases were shown to be irreversibly inhibited by aspirin as a result of acetylation of a serine residue (Ser-530 and Ser-516 for COX-1 and COX-2, respectively) located at the binding site of arachidonic acid, the endogenous substrate of cyclooxygenases (Lecomte et al., J. Biol. Chem., 269:13207-13215 (1994)). In the present study, it was initially discovered that NAA at physiological concentrations caused a significant decrease in prostaglandin E₂ (PGE₂) produced by ionomycin-stimulated STTG astroglial cells. As expected, aspirin had the same effect. Therefore, the initial hypothesis focused on the potential acetylation of COX-2 by NAA, similar to the mechanism of inhibition of aspirin. Glial cells are able to breakdown NAA into acetate and aspartate by the action of aspartoacylase (Chakraborty et al., J. Neurochem., 78:736-745 (2001)). This acetate could bind to the active site of COX-2.

Exogenous PGE₂ administration to colon cells has been shown to enhance carcinogenesis and reduce apoptosis (Kawamori et al., Carcinogenesis, 24:985-990 (2003)). COX-derived prostaglandins have also been shown to increase vascular endothelial growth factor (VEGF) and thereby promote angiogenesis in cancer cells (Lim, Oncol. Rep., 10: 1241-1249 (2003)). Also, COX-2 is significantly induced in astrocyte and microglial cultures by radiation injury (Kyrkanides et al., Brain Res. Mol. Brain. Res., 104:159-169 (2002)). Potential roles for NAA in the brain could be as an anti-proliferation, anti-angiogenic, anti-inflammatory molecule through the control of the amount of prostaglandins produced.

Prostaglandins have very important physiological roles in the central nervous system. In addition to promoting adequate perfusion (Bentzer et al., J. Neurotrauma, 20:447-461 (2003)), prostaglandins also are important for neuronal signaling within the brain (Rage et al., J. Neurosci., 17:9145-9156 (1997)). In this current investigation, high levels of NAA (20 mM) caused a complete inhibition (>95%) of prostaglandin release in STTG cells. Therefore, in pathological conditions in which NAA levels are elevated in the brain (i.e., Canavan's disease), the elevated levels of NAA might be detrimental to normal neuronal function by completely eliminating prostaglandin production. In Canavan's disease, a progressive spongy degeneration of the white matter of the brain is the hallmark physiological sign of this genetically inherited disease (Matalon et al., Front Biosci., 5:D307-D311 (2000)). This degeneration involves the loss of the axon's myelin sheath (Baslow et al., J. Mol. Neurosci., 9:109-125 (1997)).

The possibility of NAA acting as an antagonist of the glutamate receptor was studied indirectly by assessing the ability of known glutamate receptor antagonists such as L-AP-4 and L-glutamic acid diethyl ester (GDE) to inhibit PGE₂ formation. Both glutamate receptor antagonists inhibited the formation of PGE₂ in the present study. Akimitsu et al. (Brain Res., 861:143-150 (2000)) demonstrated that NAA-induced seizures are antagonized by GDE, suggesting that NAA acts on glutamate receptors. The calcium ionophore ionomycin, used as a stimulant in this current study, has been shown by Jeftinija et al. (J. Neurochem., 66:676-684 (1996)) to cause a calcium-dependent release of excitatory amino acids such as glutamate from neocortical astrocytes. Binding of glutamate to receptors on the astrocyte results in astrocyte activation (Porter et al., J. Neurosci., 16:5073-5081 (1996)) and release of arachidonic acid from astrocytes (Stella et al., J. Neurosci., 14:568-575 (1994)). Based on the findings in the present study and the findings of others, NAA possibly acts on glutamate receptors of astrocytes thereby limiting cell activation and subsequent prostaglandin formation.

Another finding in the present study was the effect that NAA had on total COX-2 protein in STTG cells. In unstimulated STTG cells, NAA did not have an effect on total, resting COX-2 protein levels. Once the cells were stimulated with IL-1β, NAA decreased the amount of total, induced COX-2 protein levels. John and coworkers (Proc. Natl. Acad. Sci. USA, 96:11613-11618 (1999)) have shown that IL-11 potentiates the transmission of intercellular calcium waves in primary human fetal astrocytes. Calcium is a very important secondary messenger in signal transduction and cell-to-cell signaling. Indeed, in astrocytes, an increase in intercellular calcium levels leads to a graded release of the excitatory amino acid glutamate as demonstrated by Parpura and Haydon (Proc. Natl. Acad. Sci. USA, 97:8629-8634 (2000)). Glutamate is then able to bind to its receptor on the neuron to initiate a synaptic transmission. Intercellular calcium chelation was shown by Grohn and Kauppinen (Cell Calcium, 20:509-514 (1996)) to prevent cell damage following severe hypoxia in the cerebral cortex. Interestingly, NAA can chelate calcium at a 1:1 ratio by means of the two, negatively charged carboxylic groups located on the NAA molecule at physiological pH (Rubin et al., J. Inorg. Biochem., 60:31-43 (1995)). Therefore, in relation to the present experimental findings, a potential mechanism explaining the decrease in COX-2 protein levels in STTG cells stimulated by IL-1β could be the ability of NAA to chelate calcium, an important secondary cell messenger.

The chelation of calcium by NAA is also crucial to the decrease in production of PGE₂ measured in STTG cells. For the PGE₂ experiment, we stimulated STTG cells with ionomycin. Venance et al. (J. Neurosci., 17:1981-1992 (1997)) showed that the presence of external calcium was necessary for induction of intercellular calcium waves by ionomycin in cultured astrocytes. Therefore, the ability of NAA to chelate extracellular calcium could contribute to the decrease in PGE₂ levels detected in our experimental system. This decrease in PGE₂ is important since PGE₂ can act as a stimulant causing an elevation in calcium levels within astrocytes resulting in the release of glutamate (Bezzi et al., Nature, 391:281-285 (1998); Sanzgiri et al., J. Neurobiol., 41:221-229 (1999)).

In addition to decreasing PGE₂ levels, another similarity between NAA and aspirin is that NAA was able to decrease the amount of activated NFκB in STTG cells stimulated with IL-1β. Multiple sources have demonstrated that aspirin inhibits NFκB activation by acetylating and inactivating the kinase responsible for phosphorylating the inhibitory subunit of NFκB, IκBα (Schwenger et al., Mol. Cell. Biol., 18:78-84 (1998); Muller et al., FASEB J, 15:1822-1824 (2001); Murono et al., Cancer Res., 60:2555-2561 (2000)). Once IκBα is phosphorylated it is degraded rapidly thereby releasing activated NFκB. Whether this decrease in activated NFκB in STTG cells caused by NAA is due to enzyme acetylation (i.e., COX-2 or IκBα kinase), calcium chelation, and/or glutamate receptor antagonism is currently under investigation. Calcium chelation seems to be the more attractive mechanism since an increase in intercellular calcium is an early event in the inflammatory, signal transduction pathway. Both NFκB activation and glutamate release are downstream from calcium fluxes in the astrocyte. Prostaglandin production also is downstream since IL-11 stimulation causes an 8-fold increase in COX-2 protein levels in endothelial cells (Camacho et al., J. Biol. Chem., 270:17279-17286 (1995)). Down regulation of activated NFκB in brain may in turn lead to decreases in pro-inflammatory cytokines such as TNFα, IL-1, and IL-6 (Friedman et al., J. Biol. Chem., 271:31115-31120 (1996); Nomoto et al., Neurosurgery, 48:158-166 (2001); Parker et al., Br. J. Pharmacol., 136:312-320 (2002)).

The results of the present study demonstrate that there are potentially other important roles for N-acetyl aspartate. In addition to being an acetyl donor for myelin synthesis and a regulator of osmosis, NAA appears to be important in the modulation of inflammation within the central nervous system. The data from the present study clearly demonstrate the decrease in key inflammatory components such as prostaglandin E₂, COX-2, and NFκB in stimulated STTG astroglial cells when NAA is present. When declines in NAA concentrations are observed in various neurological pathologies, common signs and symptoms would indicate inflammation. Accordingly, abnormally low NAA levels in diseases such as Huntington's disease, ischemic injuries, etc., are possibly due to over utilization of NAA to counteract inflammatory pathways. In Canavan's disease, the large concentrations of NAA result in neuronal loss possibly due to the complete shutdown of key components of normal cell function (i.e., homeostatic prostaglandin production, cell-to-cell signaling, etc.) The findings of the present study shed some light into the biological rationale for the presence of millimolar concentrations of NAA in certain compartments of the central nervous system and lend some insight into why disruption of these levels may lead to some of the associated neurological deficits observed.

1-62. (canceled)

63. A method of treating inflammation of a tissue or an organ of an animal comprising administering to the animal an effective amount of a compound of the formula:
R¹—C(O)—NH—CH₂(CH₂—COOR²)—COOR²
wherein:
R¹ is H, a lower alkyl or a lower alkyl substituted with a halogen atom; and
R², each of which may be the same or different, is H or an alkyl, cycloalkyl, aryl, alkylaryl
or arylalkyl, each of which may optionally be substituted with a polar substitutent;
or a pharmaceutically-acceptable salt thereof.

64. The method of claim 63 wherein the inflammation is inflammation of a mouth tissue, a mucous membrane, a portion of the respiratory system or a portion of the gastrointestinal tract of the animal.

65. The method of claim 64 wherein the inflammation is inflammation of a portion of the gastrointestinal tract of the animal.

66. The method of claim 64 wherein the inflammation is inflammation of a portion of the respiratory system of the animal.

67. The method of claim 64 wherein the inflammation is inflammation of a mouth tissue of the animal.

68. The method of claim 64 wherein the inflammation is inflammation of a mucous membrane of the animal.

69. A method of treating an inflammatory disease or condition of an animal other than an inflammatory disease or condition of the animal's skin, the method comprising administering to the animal an effective amount of a compound of the formula:
R¹—C(O)—NH—CH₂(CH₂—COOR²)—COOR²
wherein:
R¹ is H, a lower alkyl or a lower alkyl substituted with a halogen atom; and
R², each of which may be the same or different, is H or an alkyl, cycloalkyl, aryl, alkylaryl
or arylalkyl, each of which may optionally be substituted with a polar substitutent;
or a pharmaceutically-acceptable salt thereof.

70. The method of claim 69 wherein the disease or condition is an inflammatory disease or condition of the mouth, the respiratory system or the gastrointestinal tract of the animal.

71. The method of claim 70 wherein the disease or condition is an inflammatory disease or condition of the respiratory system of the animal.

72. The method of claim 71 wherein the disease or condition is acute respiratory distress syndrome, asthma, bronchitis, emphysema, pulmonary fibrosis or a respiratory system infection.

73. The method of claim 70 wherein the disease or condition is an inflammatory disease or condition of the gastrointestinal tract of the animal.

74. The method of claim 73 wherein the disease or condition is colitis, Crohn's disease, gastritis or inflammatory bowel disease.

75. The method of claim 70 wherein the disease or condition is an inflammatory disease or condition of the mouth of the animal.

76. The method of claim 75 wherein the disease or condition is gingivitis, periodontitis or an infection.

77. A pharmaceutical composition formulated for administration to an animal other than by topical administration to the animal's skin, the composition comprising a pharmaceutically-acceptable carrier and a compound of the formula:
R¹—C(O)—NH—CH₂(CH₂—COOR²)—COOR²
wherein:
R¹ is H, a lower alkyl or a lower alkyl substituted with a halogen atom; and
R², each of which may be the same or different, is H or an alkyl, cycloalkyl, aryl, alkylaryl
or arylalkyl, each of which may optionally be substituted with a polar substitutent;
or a pharmaceutically-acceptable salt thereof.

78. The composition of claim 77 which is formulated for oral administration of the compound or pharmaceutically-acceptable salt thereof.

79. The composition of claim 77 which is formulated for parenteral administration of the compound or pharmaceutically-acceptable salt thereof.

80. The composition of claim 77 which is formulated for local administration of the compound or pharmaceutically-acceptable salt thereof to a tissue of the animal's mouth.

81. The composition of claim 77 which is formulated for administration of the compound or pharmaceutically-acceptable salt thereof by inhalation.

82. The composition of claim 77 which is formulated for nasal administration of the compound or pharmaceutically-acceptable salt thereof.

83. The composition of claim 77 which is formulated for administration of the compound or pharmaceutically-acceptable salt thereof ocularly.

84. The composition of claim 77 which is formulated for administration of the compound or pharmaceutically-acceptable salt thereof vaginally or rectally.

85. The method of any one of claims 63-84 wherein the compound is N-acetyl-L-aspartic acid or a pharmaceutically-acceptable salt thereof.


SEQUENCE LISTING (2 sequences)

SEQ ID NO: 1 - DNA, 23 bp, Artificial
  This artificial sequence is a primer.
tcttttaatgagtaccgcaaacg

SEQ ID NO: 2 - DNA, 26 bp, Artificial
  This artificial sequence is a COX-2 reverse primer.
ttagacttctacagttcagtcgaacg